(19) United States
(12) Patent Application Publication
(10) Pub. No.: US 2023/0110824 A1
(43) Pub. Date: Apr. 13, 2023

(54) FLUORESCENT PROBES FOR DETECTION OF CALCIFICATIONS

(71) Applicant: TECHNISCHE UNIVERSITÄT BERLIN, Berlin (DE)

(72) Inventors: Gündog Yücesan, Berlin (DE); Hajo Haase, Berlin (DE)

(73) Assignee: TECHNISCHE UNIVERSITÄT BERLIN, Berlin (DE)

(21) Appl. No.: 17/802,759

(22) PCT Filed: Feb. 26, 2021
(86) PCT No.: PCT/EP2021/054792

§ 371 Date: Aug. 26, 2022

(30) Foreign Application Priority Data
Feb. 28, 2020 (EP) 20160098.8

(51) Int. Cl. G01N 33/58; G01N 33/84; C09K 11/06; G01N 33/574; G01N 21/64

(52) U.S. Cl. CPC G01N 33/582; G01N 33/84; C09K 11/06; G01N 33/57415; G01N 21/6428; G01N 2021/6439; G01N 2800/365; G01N 2223/10; G01N 2800/105

(57) ABSTRACT

A fluorescent probe includes one or more metal binding functional group, such as phosphonic acid group and an arsonic acid group, in which the functional group is covalently linked to a fluorescent core via a sp²-carbon atom of the fluorescent core. In embodiments, the fluorescent core is an organic fluorescent compound/moiety, that can be a tetrapyrrole derivative, such as porphyrin or phthalocyanine, acridine, BODIPY, cyanine or cyanine derivatives, carbazole, coumarine or coumarine derivatives, xanthene or xanthene derivatives such as fluorescein or rhodamine. The fluorescent probe can bind to calcium and/or a calcification, such as hydroxyapatite (HAP). In a further aspect, a fluorescent probe is used in a method of detecting calcium, such as a calcification or HAP, in a bodily tissue. The use of the fluorescent probe is also provided for detecting calcium, a calcification and/or HAP, such as calcium depositions in a bodily tissue.

The invention relates to a fluorescent probe comprising one or more metal binding functional group, preferably selected the group comprising phosphonic acid group and an arsonic acid group, wherein the functional group is covalently linked to a fluorescent core via a sp²-carbon atom of the fluorescent core. In embodiments, the fluorescent core is an organic fluorescent compound/moiety, preferably a tetrapyrrole derivative, such as porphyrin or phthalocyanine, acridine, BODIPY, cyanine or cyanine derivatives, carbazole, coumarine or coumarine derivatives, xanthene or xanthene derivatives such as fluorescein or rhodamine. Preferably, the fluorescent probe of the invention can bind to calcium and/or a calcification, such as preferably hydroxyapatite (HAP). In a further aspect, the invention relates a fluorescent probe of the invention for use in a method of detecting calcium, preferably a calcification or HAP, in a bodily tissue. Also, the invention relates to the use of the fluorescent probe of the invention for detecting calcium, a calcification and/or HAP, preferably calcium depositions in a bodily tissue.

### BACKGROUND OF THE INVENTION

Dysregulation of calcium and phosphate homeostasis often leads to the pathological deposition of minerals such as calcium carbonate (CC), calcium oxalate (CO), calcium phosphate (CP), calcium pyrophosphate (CPP), and hydroxyapatite in many soft tissues, such as those in the brain, eye, kidney and skin. The pathological deposition of these minerals causes these tissues to become inflexible and brittle and will prevent the passive diffusion of molecules between cells and their environment. In addition, minerals like HAP can facilitate the retention of molecules by providing a binding surface for the pathological accumulation of molecules (https://www.ncbi.nlm.nih.gov/pubmed/25605911). Monitoring the increase in calcifications can be a valuable early indication of breast cancer while monitoring the decrease in mineralization is useful for osteoporosis. Therefore, monitoring calcification in health and disease is critical for diagnosis and treatment.

There are several methods that are used to monitor calcifications, some work in vivo while others only work in vitro. A number of radiological techniques have the potential to detect calcification using radiography, fluoroscopy, conventional computed tomography (CT), electron-beam tomography (EBT), multi-detector CT, intravascular ultrasound, magnetic resonance imaging (MRI), and transthoracic and transesophageal echocardiography. However, most current instruments cannot resolve smaller than millimeter sized deposits or the measurement needs to be invasive like that of optical coherence tomopraphy (˜10 um resolution) (Ying Wang et al., “Imaging Cardiovascular Calcification”, J Am Heart Assoc. 2018 Jul. 3; 7(13): e008564, PMCID: PMC6064897). In vitro, the resolution can be significantly increased. The classic Von Kossa histochemical method relays on silver precipitation at sites of phosphate deposition while Alizarin red, a calorimetric label, is widely used to detect calcium deposition. These are effective, inexpensive and widely used methods, but they are not very sensitive.

Fluorescent probes broadly offer better sensitivity than colorimetric ones, but existing stains such as Alizarin Red S, Xylenol Orange, or uranium have clear drawbacks such as modest selectivity among mineral phases, pH dependence, overlap of their fluorescence with tissue autofluorescence, and other issues.

Since light scattering, absorbance, and tissue autofluorescence at visible wavelengths make it difficult to use such probes in vivo even in small animal models (“Red and Near-Infrared Fluorometry”: R. B. Thompson in Topics in Fluorescence Spectroscopy Vol. 4: Probe Design and Chemical Sensing (Eds.: J. R. Lakowicz), 1994, New York, Plenum Press, pp. 151-181.), the Frangioni and Kovar groups developed second generation fluorescent probes coupling HAP-binding moieties such as bisphosphonate or tetracycline with red and near-infrared penta- or hepta-methine cyanine fluorescent moieties (J. L. Kovar, X. Xu, D. Draney, A. Cupp, M. A. Simpson, D. M. Olive, Anal. Biochem. 2011, 416, 167-173). These probes were sensitive, selective, and usable in vivo in animal models, but are costly and by comparison with other probes that exhibit significant or unique changes in fluorescence when binding to particular targets (R. F. Chen, J. C. Kernohan, J. Biol. Chem. 1967, 242, 5813-5823; R. H. Conrad, J. R. Heitz, L. Brand, Biochemistry 1970, 9, 1540-1546. J.-B. LePecq, C. Paoletti, J. Mol. Biol. 1967, 27, 87-106) their fluorescence is insensitive to whether they have bound to the (calcified) target or nonspecifically to something else.

Phosphonate chemistry has recently attracted significant attention in diverse scientific fields ranging from material science to medicine [1-2]. Initial toxicity studies with aromatic phosphonic acids and phosphonate metal-organic solids were promising for their in vivo applications in as much as p-H₈TPPA exhibited no toxicity for an intestinal cell line at high concentrations. [9, 10] One of the unexplored properties of phosphonic acid metal binding groups is their potential role in imaging free metal ions, or metal deposits in living systems.

Recent work, and Martell's early work from the 1970s all indicate that phosphonates exhibit high affinity towards alkali and alkaline earth metal ions.[10, 12, 13, 14] In addition, the pKa₂ of phenyl phosphonic acid (PhPO₃H₂) is 7.44, giving PhPO₃²⁻ a −2 charge under physiological pH. Therefore, the use of phosphonic acid metal binding unit(s) to target calcium ions and calcifications offers highly sensitive and pH independent metal probes working near physiological pH.

Bisphosphonates have been previously attached to the cyanine and fluorescein moieties with long aliphatic hydrocarbon side chains to label target calcification with near infrared fluorescence. [14, 17] The presence of sp3 bonds in the aliphatic linker chain in these examples of the state of the art between the phosphonic acid and the fluorescent core merely used bisphosphonates as recognition moieties and minimized any electronic interactions between the fluorescent core and the phosphonic acid metal binding unit. Therefore, such fluorophores did not provide any change in fluorescent intensity upon HAP binding.

To address these drawbacks of the prior art, the present invention is directed to fluorescent probes for detecting calcification bearing phosphonic acid metal binding units which are bonded to one of the sp2 carbon atoms of the fluorescent cores, with the goal of differentially perturbing the fluorescence of the probe when bound to calcified substrates, and thereby providing additional information. The use of such fluorophores would be important with respect to generating synergic interactions between the fluorescent core and the d orbitals of the target divalent metal ions.

### SUMMARY OF THE INVENTION

In light of the prior art the technical problem underlying the present invention is to provide alternative and improved means for the detection of calcifications.

This problem is solved by the features of the independent claims. Preferred embodiments of the present invention are provided by the dependent claims.

The invention relates to several aspects. Any specific embodiment or feature of the invention that is disclosed in the context of one aspect of the invention is herewith also disclosed in the context of any other aspect of the invention. Any compound or molecule disclosed herein that fulfils the requirements/features of claim 1 is a fluorescent probe and represents a preferred embodiment of the invention.

In a first aspect, the invention relates to a fluorescent probe comprising
one or more metal binding functional group, preferably selected from the group comprising phosphonic acid group and arsonic acid group,
and a fluorescent core,
wherein the one or more metal binding functional group is covalently linked to a sp or a sp²-carbon atom or a nitrogen atom of the fluorescent core.

Preferably, the one or more metal binding functional group is covalently linked to a sp or a sp²-carbon atom or a nitrogen atom of the fluorescent core via a P or As atom. Preferably, the link of the metal binding function group to the fluorescent core is via a sp²-carbon atom via a P or As atom.

The present invention is based on the entirely surprising discovery that the fluorescent probes of the invention, comprising a series of novel organophosphonate fluorophores, can selectively bind bone structure and microcalcifications, which lead to changed fluorescent properties of the probes that can be detected. In particular, it was found that upon binding to calcium or calcifications, such as HAP, lead to changes in the ground and excited states resulting in quenching or enhancing the fluorescence emission. It is suspected that the metal binding groups, in particular phosphonic acid and arsonic acid groups, having direct sp² bonds to the fluorescent core can extend the conjugation of the fluorescent core to the HAP and thereby initiate such changes. Such properties of the fluorescent probes of the invention can be used to monitor bone growth, resorption and microcalcification formation, which is an early indication of breast cancer.

Currently, Perkin Elmer has a commercial product called osteosense 680 to monitor such properties. Comparative studies with osteosense indicated that the fluorescent probes of the invention are much cheaper to produce via Suzuki Cross Coupling reactions, provide clearer imaging results, and a larger fluorescence range. In addition, the probes of the invention work with much lower concentrations and with better efficiency. These probes can also be used in monitoring drusen formation to detect early stages of age-related macular degeneration disease.

Importantly, all the organophosphonate fluorophores disclosed herein are novel except p-H₈TPPA and m-H₈TPPA or with TPPA core, and the calcium sensing properties of the probes have not been reported neither in patent nor scientific literature.

Furthermore, the examples provided herein, including experiments with the intestinal Caco-2 cell line, indicate that the fluorescent probes of the invention, and in particular those based on a porphyrine core, are well tolerated by the intestinal cell line.

The present invention is based on the unexpected idea that a calcium sensing unit (metal binding functional group) of the probe of the invention should be connected to the organic fluorescent core directly via at least one of the sp or sp²-carbon atoms and/or via at least one nitrogen atom to produce extended conjugation interacting with the target metal. In embodiments, the metal binding functional group being selected from the group comprising or consisting of a phosphonic acid group and an arsonic acid group are covalently linked to a sp or a sp²-carbon atom or a sp or a sp²-nitrogen atom of the fluorescent core via a P or As atom.

In the context of the invention, it is understood that an organic fluorescent core is a fluorescent organic molecule/structure comprising one or more (combined) aromatic groups, and/or planar or cyclic structures with several π bonds, which form a conjugated system of delocalized electrons.

As a consequence, interaction of a probe of the invention with calcium, a calcium deposition or HAP, via the metal binding group affects the conjugated system of electrons resulting in a modification of the fluorescent properties of the fluorescent core, since the conjugated system extends to the metal binding group.

In contrast, sp³ bonded metal sensing units that are connected to a fluorescent core can mediate binding of such a probe to a metal ion but binding of the metal ion does not influence the fluorescent properties of the probe, at least not to the same extend as it is the case for a probe of the method presented herein, since the conjugated electron system is not in contact with the metal binding group and the binding metal ion.

In embodiments of the invention, the metal binding group is connected to the fluorescent core via phenylphosphonic acid tethers. Phenylphosphonic acid tethers provide 1.7 and 7.4 pKa1 and pKa2 values, respectively, and each of the phenylphosphonic acid tethers are expected to provide −2 negative charge at physiological pH. Therefore, phosphonic acid derivatives are suitable to generate ionic interactions with divalent calcium ions in biological systems.

In embodiments of the invention, the one or more metal binding functional group is selected from the group comprising a phosphonic acid group and an arsonic acid group and is covalently linked to a sp²-carbon atom or a nitrogen atom of the fluorescent core via a P or As atom.

In embodiments, the probes comprise conjugated/aromatic tethers, which can be regarded as an extension of the conjugated system of the fluorescent core to the location of the metal binding functional group, it is decisive that the metal binding groups are connected to the fluorescent core via a continuous system of delocalized electrons for enabling detectable modification of the fluorescent properties by metal ion binding. This extension of the conjugated system is brought about by binding the P or As atom of the metal binding phosphonic acid or arsonic acid group directly to an sp or sp² C or N atom of the fluorescent core, or by connecting the metal binding functional group and the sp or sp² C or N atom of the functional groups with tethers that expand the delocalized electron system of the fluorescent core to the functional metal binding group.

In embodiments, the probes of the invention are used and/or methods of the invention are performed at a pH in the range of 6-9, preferably at a pH of about 7-8, such as about 7.4. pH conditions in the range of a physiological pH are preferred, since the probes of the invention are intended for use on bodily sample, such as tissue samples, or are even envisioned to be administered to a subject, such as a human subject.

Preferably, the fluorescent probe of the invention is for detecting calcium and/or a calcification.

In embodiments, the fluorescent core of the probe of the invention is an organic fluorescent compound/moiety, preferably a tetrapyrrole derivatives, such as porphyrin or phthalocyanine, acridine, BODIPY, cyanine or cyanine derivatives, carbazole, coumarine or coumarine derivatives, xanthene or xanthene derivatives such as fluorescein or rhodamine.

It surprisingly turned out that the concept of connecting the metal binding functional group directly to a sp² or sp carbon or nitrogen atom that forms part of the delocalized electron system of the fluorescent core is applicable to various kinds of fluorescent cores.

In embodiments, the one or more metal binding functional group of the fluorescent probe of the invention is a phosphonic acid group.

In embodiments, the fluorescent probe comprises two or more metal binding functional groups.

In embodiments, the probe comprises at least two, three, four, five, six, seven, eight, nine or ten or more metal binding functional groups.

It was surprisingly found out that the inclusion of two metal binding groups as compared to only one is advantageous, since upon binding of calcium or calcium salts, such as calcium depositions or HAP, the detectable change of fluorescence increases in comparison to a corresponding probe with only one metal binding group. Furthermore, it was found that the probes bind stronger to the calcifications present in a tissue sample or tissue and therefore allow a more specific and sensitive detection of depositions. This holds also true for embodiments where two metal binding groups of a probe can bind to the same calcium. Accordingly, the use of probes with more metal binding groups is advantageous. Therefore, in preferred embodiments, the probes comprise 2, 3, 4, 5, 6, 7, 8 or more metal binding functional groups, such as phosphonic acid groups and/or arsonic acid groups. In embodiments, such an increase in the number of metal binding phosphonic acid or arsonic acid functional groups improves the sensitivity and allows fine-tuning of the affinity of the probes to calcium depositions in tissues or cells.

In embodiments of the invention, one calcium atom can bind to more than one metal binding groups, such as to two metal binding groups, wherein the different metal binding groups bound by one calcium atom, can be comprised in the same probe molecule or can be of different probe molecules.

In embodiments, the fluorescent probe of the invention comprises or consists of a compound selected from the group comprising
((4-(5,5-difluoro-1,3,7,9-tetramethyl-5H-414,514-dipyrrolo[1,2-c:2′,1′-f][1,3,2]diazaborinin-10-yl)phenyl)phosphonic acid) (BODIPY-PPA),
5,10,15,20-tetrakis[p-phenylphosphonic acid] porphyrin (p-H₈TPPA), and
5,10,15,20-tetrakis[m-phenylphosphonic acid] porphyrin (m-H₈TPPA).

As shown in the examples below, BODIPY-PPA, p-H8TPPA and m-H8TPPA can be used for the specific detection of calcification in tissue and can even change their fluorescent properties upon binding calcifications. Accordingly, the probes are suitable for detection of calcium depositions in tissue and can be used for various diagnostic and medical purposes.

In embodiments, measuring fluorescence is performed by exciting the sample with light of an excitation wavelength and detecting emitted light at an emission wavelength. Accordingly, it is possible to detect the presence of calcifications and calcium depositions by performing standard fluorescence measurements using diagnostic and medical equipment known to the skilled person.

In preferred embodiments, the probe binds to calcium and/or a calcification, such as preferably hydroxyapatite (HAP), wherein the calcium, the calcification and/or the HAP may be deposited in a bodily tissue. In embodiments, the probe of the invention can bind to calcium and/or a calcification.

In preferred embodiments of the invention, binding of the fluorescent probe to calcium, a calcification or HAP leads to an increase in fluorescence, such as an increase in emission (of light) by the probe upon stimulation.

It is a great advantage of the probes of the invention that upon binding to calcifications the fluorescence is increasing, since this enables specific detection of calcifications in body tissues with a high specificity and sensitivity, even if only very small calcification particles are present in the respective tissue. Accordingly, the use of the probes of the invention can lead to an important improvement of current imaging techniques used for the detection of calcifications in tissue due to the specific binding and possible high contrast generated by the probes.

In another aspect, the invention relates to the use of a fluorescent probe of the invention as a contrast agent. The probes can be comprise by a contrasting agent, which can be administered to a patient or subject before performing an imaging of the subject's body, for example for diagnostic or analytic purposes.

The invention further relates to a contrast agent, comprising a fluorescent probe of the invention. A contrasting agent can be a liquid or a table or any other kind of known an useful composition that can be administered to a subject or sample. For example, routes for administering contrasting agents can be oral administration, intravenous administration, intraperitoneal administration, among other known routes generally used in the context of various imaging techniques.

In case of in vitro analysis of sample, for example tissue sample or other bodily samples, for example bodily fluids such as blood, serum, plasma, urine or tears; feces; homogenized or liquified tissue samples or cells, the probe can be added to the sample in a suitable buffer solution. Furthermore, the probe or a contrasting agent may be directly injected into a tissue, for example in a buffer solution.

In another aspect, the invention relates to the use of a fluorescent probe of the invention for detecting calcium, a calcification and/or HAP.

The invention further relates to the use of a fluorescent probe of the invention in a method of detecting calcium, a calcification and/or HAP, preferably in a bodily tissue.

Furthermore, the invention relates to the use of a fluorescent probe of the invention for detecting a calcium deposition, such as a calcification or HAP, in a bodily tissue.

In the context of the use of the fluorescent probe for detecting a calcium deposition in a bodily tissue, the tissue is a soft tissue, such as brain, eye, kidney, skin, gastrointestinal organs, liver, organs, tendons, ligaments, fascia, skin, fibrous tissues, fat, synovial membranes, muscles, nerves or blood vessels.

Tissue calcification can be part of pathological processes and therefore early, specific and sensitive detection can be important for diagnosis and early detection of pathological processes. Also, in cases where calcification is a physiological process, for example during bone, cartilage and teeth generation or regeneration, tissue growth, remodeling and/or regeneration can be monitored by means of the probes of the invention. The other was around, degeneration of such processes can also be observed and monitored.

Therefore, preferably, the fluorescent probe is used for detecting bone growth and/or resorption.

Furthermore, the probe can also be used for detecting breast calcification/microcalcifications in breast tissue. Breast calcifications can be an early sign of breast cancer and therefore early detection, in particular of microcalcifications, can be part of early detection and screenings for breast cancer.

In another aspect, the invention relates to the fluorescent probe of the invention for use in a diagnostic method.

Preferably, the diagnostic method is a method of detecting calcifications in a bodily tissue.

In embodiments, the diagnostic method is method of detecting breast cancer.

In embodiments, a method or diagnostic method comprising the use of the fluorescent probe of the invention can comprise the steps of
contacting the tissue, lumen or cell with a fluorescent probe of the invention,
irradiating the tissue, lumen, or cells at a wavelength absorbed by the compound;
and detecting a signal from the fluorescent probe, thereby imaging the tissue, lumen, or cells.

In a further aspect, the invention relates to a method of imaging tissue, lumens, or cells, the method comprising
contacting the tissue, lumen or cell with a fluorescent probe of the invention,
irradiating the tissue, lumen, or cells at a wavelength absorbed by the compound;
and detecting a signal from the fluorescent probe, thereby imaging the tissue, lumen, or cells.

In embodiments, the methods and uses employing the probe of the invention are in vitro methods, preferably using isolated sample, such as tissue samples isolated from a subject.

In embodiments, a probe of the invention is administered to a subject, such as a patient suspected of having calcium depositions in a bodily tissue, for in vivo imaging.

In embodiments, the imaging agent is administered to an organism comprising the tissue, lumen, or cells. Preferably, the organism is a mammal, most preferably a human.

Preferably, the tissue, lumen or cell comprise bone cells comprise, cartilage cells and/or their products. Accordingly, the invention can relate to a method of imaging bone cells and/or cartilage cells and/or their products, the method comprising
contacting the bone cells, cartilage cells and/or their products with a fluorescent probe of the invention,
irradiating the tissue at a wavelength absorbed by the imaging agent;
and detecting a signal from the fluorescent probe, thereby imaging the bone cells, cartilage cells and/or their products.

In embodiments, the invention relates to a fluorescent probe of the invention as described herein for use in a method of the invention as described herein.

### DETAILED DESCRIPTION

The present invention relates to a fluorescent probe comprising one or more metal binding functional group, preferably selected from the group comprising phosphonic acid group and arsonic acid group, and a fluorescent core, wherein the one or more functional group is covalently linked to a sp or (preferably) a sp²-carbon atom of the fluorescent core.

In the context of the invention, the term “fluorescent probe” refers to a fluorescent molecule (also called fluorophore) whose fluorescence is affected by environmental aspects such as polarity or ions, in case of the present invention by binding of metal ions to the metal binding functional group. A fluorophore is a fluorescent chemical compound that can re-emit light upon light excitation. Fluorophores typically contain several combined aromatic groups, or planar or cyclic molecules with several π bonds. Fluorophores can be used alone, as a tracer in fluids, as a dye for staining of certain structures, as a substrate of enzymes, or as a probe or indicator, when its fluorescence is affected by environmental aspects, such as binding of a ligand to the fluorophore. Importantly, fluorophores and fluorescent probes can be used to stain tissues, cells, or materials in a variety of analytical methods, i.e., fluorescent imaging, microscopy and spectroscopy.

Fluorescence is the emission of light by a substance that has absorbed light or other electromagnetic radiation. It is a form of luminescence. In most cases, the emitted light has a longer wavelength, and therefore lower energy, than the absorbed radiation. The most striking example of fluorescence occurs when the absorbed radiation is in the ultraviolet region of the spectrum, and thus invisible to the human eye, while the emitted light is in the visible region, which gives the fluorescent substance a distinct color that can be seen only when exposed to UV light. Fluorescent materials cease to glow nearly immediately when the radiation source stops, unlike phosphorescent materials, which continue to emit light for some time after.

Fluorescence is brought about by absorption of photons in the singlet ground state promoted to a singlet excited state. The spin of the electron is still paired with the ground state electron, unlike phosphorescence. As the excited molecule returns to ground state, it involves the emission of a photon of lower energy, which corresponds to a longer wavelength, than the absorbed photon.

The fluorescent probes that are used in the context of the present invention comprise an organic fluorescent core and one or more metal binding functional groups.

As used herein, the term organic fluorescent core refers to a fluorescent organic molecular structure comprising delocalized electronic structure. Delocalized electrons are electrons in a molecule that are not associated with a single atom or a covalent bond. In organic chemistry, the term delocalization refers to resonance in conjugated systems and aromatic compounds.

A conjugated system is a system of connected p orbitals with delocalized electrons in a molecule, which in general lowers the overall energy of the molecule and increases stability. It is conventionally represented as having alternating single and multiple bonds. Lone pairs, radicals or carbenium ions may be part of the system, which may be cyclic, acyclic, linear or mixed. Conjugation is the overlap of one p orbital with another across an intervening σ bond. A conjugated system has a region of overlapping p orbitals, bridging the interjacent locations that simple diagrams illustrate as not having a π bond. They allow a delocalization of π electrons across all the adjacent aligned p orbitals. The π electrons do not belong to a single bond or atom, but rather to a group of atoms.

Aromatic structures are cyclic (ring-shaped) and planar (flat) with a ring of resonance bonds that gives increased stability compared to other geometric or connective arrangements with the same set of atoms. Aromatic molecules are very stable, and do not break apart easily to react with other substances. Organic compounds that are not aromatic are classified as aliphatic compounds—they might be cyclic, but only aromatic rings have special stability (low reactivity). In terms of the electronic nature of the molecule, aromaticity describes a conjugated system often made of alternating single and double bonds in a ring. This configuration allows for the electrons in the molecule's pi system to be delocalized around the ring, increasing the molecule's stability. The molecule cannot be represented by one structure, but rather a resonance hybrid of different structures, such as with the two resonance structures of benzene.

Conjugation is possible by means of alternating single and double bonds in which each atom supplies a p orbital perpendicular to the plane of the molecule. However, that is not the only way for conjugation to take place. As long as each contiguous atom in a chain has an available p orbital, the system can be considered conjugated. For example, furan is a five-membered ring with two alternating double bonds flanking an oxygen in a five-membered ring. Oxygen has two lone pairs, one of which occupies a p orbital perpendicular to the ring on that position, thereby maintaining the conjugation of that five-membered ring by overlap with the perpendicular p orbital on each of the adjacent carbon atoms. The other lone pair remains in plane and does not participate in conjugation.

In general, any sp² or sp-hybridized carbon or heteroatom, including ones bearing an empty orbital or lone pair orbital, can participate in conjugated systems, though lone pairs do not always participate in a conjugated system. For example, in pyridine, the nitrogen atom already participates in the conjugated system through a formal double bond with an adjacent carbon, so the lone pair remains in the plane of the ring in an sp² hybrid orbital and does not participate in the conjugation. A requirement for conjugation is orbital overlap; thus, the conjugated system must be planar (or nearly so). As a consequence, lone pairs which do participate in conjugated systems will occupy orbitals of pure p character instead of spⁿ hybrid orbitals typical for nonconjugated lone pairs.

In organic fluorescent structures, conjugated π systems absorb UV or visible light. Deletion of specific absorbed wavelengths from reflected visible light leads to the perception of color. However, a very small fraction of conjugated systems converts the absorbed energy into re-emission of light-fluorescence. Absorbance of light by a conjugated π system is the result of the energy of incoming UV and/or visible light matching the π/π* energy gap. This allows excitation of a HOMO π electron to the π* orbital (Prior to excitation, the π* orbital would be denoted as the LUMO.). This generates a high-energy (excited) state of the molecule, where one electron populates the antibonding π* orbital, and one electron remains in what was the fully-bonding π orbital.

The utility of fluorescence originates with the difference between the excitation and emission wavelengths. Because the excitation and emission wavelengths are different, emission intensity can be measured with minimized interference from the incoming excitation light, enabling to distinguish input and output.

Non-limiting examples of organic fluorescent cores that can be comprised by a probe of the invention and for use in the context of the methods described herein comprise tetrapyrrole derivatives, such as porphyrin or phthalocyanine, acridine, BODIPY, cyanine or cyanine derivatives, carbazole, coumarin or coumarin derivatives, xanthene or xanthene derivatives such as fluorescein or rhodamine.

Tetrapyrroles are a class of chemical compounds that contain four pyrrole or pyrrole-like rings. The pyrrole/pyrrole derivatives are linked by (═(CH)— or —CH₂— units), in either a linear or a cyclic fashion. Pyrroles are a five-atom ring with four carbon atoms and one nitrogen atom. Tetrapyrroles are common cofactors in biochemistry and their biosynthesis and degradation feature prominently in the chemistry of life.

Porphyrin is a particularly preferred fluorescent core of the invention. Porphyrins are a group of heterocyclic macrocycle organic compounds, composed of four modified pyrrole subunits interconnected at their a carbon atoms via methine bridges (═CH—). The parent of porphyrin is porphine, a rare chemical compound of exclusively theoretical interest. Substituted porphines are called porphyrins and can be represented by the following formula:

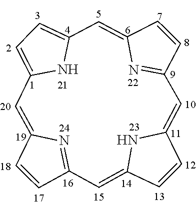 [SMILES: C1=Cc2cc3ccc(cc4nc(cc5ccc(cc1n2)[nH]5)C=C4)[nH]3]

With a total of 26 π-electrons, of which 18 π-electrons form a planar, continuous cycle, the porphyrin ring structure is often described as aromatic. One result of the large conjugated system is that porphyrins typically absorb strongly in the visible region of the electromagnetic spectrum, i.e. they are deeply colored.

Phthalocyanine (H₂Pc) is a large, aromatic, macrocyclic, organic compound with the formula (C₈H₄N₂)₄H₂ and is of specialized interest. It can be depicted by the following formula:

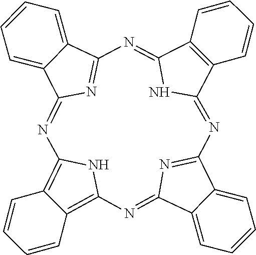 [SMILES: c1ccc2c(c1)-c1nc-2nc2[nH]c(nc3nc(nc4[nH]c(n1)c1ccccc41)-c1ccccc1-3)c1ccccc21]

Phthalocyanine is composed of four isoindole units linked by nitrogen atoms. H₂Pc has a two-dimensional geometry and a ring system consisting of 18 π-electrons. The extensive delocalization of the π-electrons affords the molecule useful properties, lending itself to applications in dyes and pigments. There are many derivatives of the parent phthalocyanine, where either carbon atoms of the macrocycle are exchanged for nitrogen atoms or the peripheral hydrogen atoms are substituted by functional groups like halogens, hydroxyl, amine, alkyl, aryl, thiol, alkoxy and nitrosyl groups. These modifications allow for the tuning of the electrochemical properties of the molecule such as absorption and emission wavelengths and conductance.

It is understood that in the context of the invention a fluorescent core can be extended to comprise aromatic tethers that extend the conjugated electron system of the base structure of the organic core. For example, in embodiments the metal binding functional group can be linked to the fluorescent core via aromatic aryl-tethers, for example in form of an arylphsphonate or arylarsonate, such as phenylphosphonate or phenylarsonate. The metal binding functional group may be linked in ortho, meta or para position of the aryl/phenyl-ring. As show in the examples below, meta and para phenylphosphonic acid groups are both compatible with porphyrin as a core structure of the fluorescent core.

In embodiments, the organic fluorescent core of the probe is acridine or derivatives thereof. Acridine is an organic compound and a nitrogen heterocycle with the formula C₁₃H₉N.

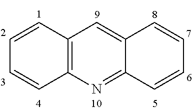 [SMILES: c1ccc2nc3ccccc3cc2c1]

Acridines are substituted derivatives of the parent ring. It is a planar molecule that is structurally related to anthracene with one of the central CH groups replaced by nitrogen. Like the related molecules pyridine and quinoline, acridine is mildly basic. It is an almost colorless solid, which crystallizes in needles. There are several commercial applications of acridines, such as the use of acridine dyes, for example acridine orange (3,6-dimethylaminoacridine).

In embodiments, the organic fluorescent core of the probe is BODIPY or derivatives thereof. BODIPY is the technical common name of a chemical compound with formula C₉H₇BN₂F₂, whose molecule consists of a boron difluoride group BF₂ joined to a dipyrromethene group C₉H₇N₂; specifically, the compound 4,4-difluoro-4-bora-3a,4a-diaza-s-indacene in the IUPAC nomenclature. The common name is an abbreviation for “boron-dipyrromethene”.

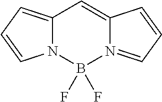

BODIPY is a red crystalline solid, stable at ambient temperature, soluble in methanol. Derivatives are obtained by replacing one or more hydrogen atoms by other functional groups and comprise the important class of BODIPY dyes. These organoboron compounds are often used as fluorescent dyes and markers in biological research.

In embodiments, the organic fluorescent core of the probe is cyanine or a cyanine derivative. Cyanines are synthetic dyes with the general formula R2N[CH═CH]nCH═N+R2↔R2N+=CH[CH═CH]nNR2 (n is a small number) in which the nitrogen and part of the conjugated chain usually form part of a heterocyclic system, such as imidazole, pyridine, pyrrole, quinoline and thiazole. Cyanines are used in industry biotechnology (labeling, analysis, biomedical imaging).

In embodiments, the organic fluorescent core of the probe is carbazole. Carbazole is an aromatic heterocyclic organic compound. It has a tricyclic structure, consisting of two six-membered benzene rings fused on either side of a five-membered nitrogen-containing ring. The compound's structure is based on the indole structure, but in which a second benzene ring is fused onto the five-membered ring at the 2-3 position of indole (equivalent to the 9a-4a double bond in carbazole, respectively).

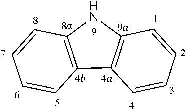 [SMILES: c1ccc2c(c1)[nH]c1ccccc12]

In embodiments, the organic fluorescent core of the probe is coumarin or a coumarin derivative. Coumarin or 2H-chromen-2-one is an aromatic organic chemical compound with the formula C₉H₆O₂. Its molecule can be described as a benzene molecule with two adjacent hydrogen atoms replaced by a lactone-like chain —(CH)═(CH)—(C═O)—O—, forming a second six-membered heterocycle that shares two carbons with the benzene ring.

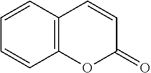 [SMILES: O=c1ccc2ccccc2o1]

Coumarin can be placed in the benzopyrone chemical class and considered as a lactone. Coumarin and its derivatives are all considered phenylpropanoids. Some naturally occurring coumarin derivatives include umbelliferone (7-hydroxycoumarin), aesculetin (6,7-dihydroxycoumarin), herniarin (7-methoxycoumarin), psoralen and imperatorin. Compounds derived from coumarin are also called coumarins or coumarinoids; this family includes: brodifacoum, bromadiolone, difenacoum, auraptene, ensaculin, phenprocoumon (Marcoumar), PSB-SB-487, PSB-SB-1202, Scopoletin (can be isolated from the bark of Shorea pinanga), warfarin (Coumadin).

In embodiments, the organic fluorescent core of the probe is xanthene or xanthene derivatives such as fluorescein or rhodamine. Xanthene (9H-xanthene, 10H-9-oxaanthracene) is the organic compound with the formula CH₂[C₆H₄]₂O.

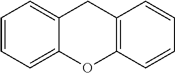 [SMILES: c1ccc2c(c1)Cc1ccccc1O2]

It is a yellow solid that is soluble in common organic solvents.

Many xanthene derivatives are useful dyes. Such xanthene dyes that contain a xanthene core include fluorescein

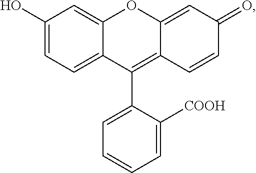 [SMILES: O=C(O)c1ccccc1-c1c2ccc(=O)cc-2oc2cc(O)ccc12]

eosins, and rhodamines

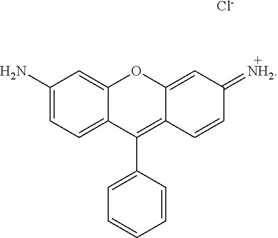

Xanthene dyes tend to be fluorescent, yellow to pink to bluish red, brilliant dyes. Many xanthene dyes can be prepared by condensation of derivates of phthalic anhydride with derivates of resorcinol or 3-aminophenol.

The term “metal binding functional group” relates to functional groups capable of binding to metal atoms, and in the context of the present invention in particular to calcium and calcium depositions, such as HAP. It is preferred that such functional groups are negatively charged acid groups that can bind metal cations. In embodiments, the functional groups of the probes of the invention are selected from the group comprising a phosphonic acid group and an arsonic acid group. In embodiments, the functional groups of the probes of the invention are selected from the group consisting of a phosphonic acid group and an arsonic acid group. In embodiments, the fluorescent probe comprises one or more phosphonic acid and/or arsonic acid groups.

Phosphonates and phosphonic acids are organophosphorus compounds containing —PO(OH)2, and phosphonicaciddiesters contain —PO(OR)2 groups (where R=alkyl, aryl). In the context of the invention, the C-atom connected to the P-Atom of the phosphonic acid is a sp or a sp²-carbon atom (C—PO(OH)2). Furthermore, in embodiments the —PO(OH)2 group may also be bound to a nitrogen atom of the fluorescent core. In embodiments, the nitrogen atom is an sp or an sp²-nitrogen atom (N—PO(OH)2).

Organophosphorus compounds are organic compounds containing phosphorus. Organophosphorus chemistry is the corresponding science of the properties and reactivity of organophosphorus compounds. Phosphorus, like nitrogen, is in group 15 of the periodic table, and thus phosphorus compounds and nitrogen compounds have many similar properties. According to one definition of organophosphorus compounds used herein, an organophosphorus compound need contain only an organic substituent, but need not have a direct phosphorus-carbon (P—C) bond. A large group of organophosphorus compounds is known to the skilled person. For example, phosphonates are esters of phosphonic acid and have the general formula RP(═O)(OR′)2; phosphate esters have the general structure P(═O)(OR)3 feature P(V); Phosphine oxides (designation σ⁴λ⁵) have the general structure R₃P═O with formal oxidation state V; Compounds with the formula [PR₄⁺]X⁻ comprise the phosphonium salts; Phosphites, sometimes called phosphite esters, have the general structure P(OR)₃ with oxidation state+3; intermediate between phosphites and phosphines are phosphonites (P(OR)₂R′) and phosphinite (P(OR)R_{′2}); the parent compound of the phosphines is PH3 or phosphane elsewhere, replacement of one or more hydrogen centers by an organic substituents (alkyl, aryl), gives PH3-xRx, an organophosphine, generally referred to as phosphines; compounds with carbon phosphorus(III) multiple bonds are called phosphaalkenes (R₂C═PR) and phosphaalkynes (RC≡P). Further example of organophohsphorus compounds are known to the skilled person.

Arsonic acids are a subset of organoarsenic compounds defined as oxyacids where a pentavalent arsenic atom is bonded to two hydroxyl groups, a third oxygen atom (this one with a double bond), and an organic substituent, which in the context of the present invention is either a C-atom (sp or sp²) or a N-atom (sp or sp²). The salts/conjugate bases of arsonic acids are called arsonates. Arsonic acid refers to H₃AsO₃, the case where the substituent is a single hydrogen atom. The other arsonic acids can simply be viewed as hydrocarbyl derivatives of this base case. Methylarsonic acid results when the substituent is a methyl group. Phenylarsonic acid results when the substituent is a phenyl group.

The probes that can be used in the method of the invention comprise at least one metal binding functional group. Preferably, the probes comprise more than one metal binding functional groups, such as 2, 3, 4, 5, 6, 7, 8, 9, 10, 11, 12, 13, 14, 15, 16, 17, 18, 19, 20 or more.

The metal binding functional group is covalently linked to a sp or sp²-carbon atom or a nitrogen atom of the fluorescent core via a P or As atom.

Orbital hybridization (or hybridization) is the concept of mixing atomic orbitals into new hybrid orbitals (with different energies, shapes, etc., than the component atomic orbitals) suitable for the pairing of electrons to form chemical bonds in valence bond theory. Hybrid orbitals are very useful in the explanation of molecular geometry and atomic bonding properties and are symmetrically disposed in space. Orbitals are a model representation of the behavior of electrons within molecules. In the case of simple hybridization, this approximation is based on atomic orbitals, similar to those obtained for the hydrogen atom, the only neutral atom for which the Schrödinger equation can be solved exactly. In heavier atoms, such as carbon, nitrogen, and oxygen, the atomic orbitals used are the 2s and 2p orbitals, similar to excited state orbitals for hydrogen.

Hybrid orbitals are assumed to be mixtures of atomic orbitals, superimposed on each other in various proportions. For example, in methane, the C hybrid orbital which forms each carbon-hydrogen bond consists of 25% s character and 75% p character and is thus described as spa (read as s-p-three) hybridized.

Spa-hybridization is explained for a tetrahedrally coordinated carbon (e.g., methane CH₄), the carbon should have 4 orbitals with the correct symmetry to bond to the 4 hydrogen atoms. Carbon's ground state configuration is 1s² 2 s² 2 p². The carbon atom can use its two singly occupied p-type orbitals, to form two covalent bonds with two hydrogen atoms, yielding the singlet methylene CH₂, the simplest carbene. The carbon atom can also bond to four hydrogen atoms by an excitation (or promotion) of an electron from the doubly occupied 2s orbital to the empty 2p orbital, producing four singly occupied orbitals. The energy released by the formation of two additional bonds more than compensates for the excitation energy required, energetically favoring the formation of four C—H bonds. Quantum mechanically, the lowest energy is obtained if the four bonds are equivalent, which requires that they are formed from equivalent orbitals on the carbon. A set of four equivalent orbitals can be obtained that are linear combinations of the valence-shell (core orbitals are almost never involved in bonding) s and p wave functions, which are the four sp³ hybrids. In CH₄, four sp3 hybrid orbitals are overlapped by hydrogen 1s orbitals, yielding four σ (sigma) bonds (that is, four single covalent bonds) of equal length and strength.

Sp²-hybridization can be explained in a similar way. For example, ethene (C2H4) has a double bond between the carbons. For this molecule, carbon sp² hybridizes, because one π (pi) bond is required for the double bond between the carbons and only three σ bonds are formed per carbon atom. In sp² hybridization the 2s orbital is mixed with only two of the three available 2p orbitals, usually denoted 2px and 2py. The third 2p orbital (2pz) remains unhybridized forming a total of three sp² orbitals with one remaining p orbital. In ethylene (ethene) the two carbon atoms form a a bond by overlapping one sp² orbital from each carbon atom. The π bond between the carbon atoms perpendicular to the molecular plane is formed by 2p-2p overlap. Each carbon atom forms covalent C—H bonds with two hydrogens by s-sp² overlap, all with 120° bond angles. The hydrogen-carbon bonds are all equal strength and length, in agreement with experimental data.

The chemical bonding in compounds such as alkynes with triple bonds is explained by sp hybridization. In this model, the 2s orbital is mixed with only one of the three p orbitals, resulting in two sp orbitals and two remaining p orbitals. The chemical bonding in acetylene (ethyne) (C₂H₂) consists of sp-sp overlap between the two carbon atoms forming a σ bond and two additional π bonds formed by p-p overlap. Each carbon also bonds to hydrogen in a σ s-sp overlap at 180° angles.

Specific examples of fluorescent probes that can be used in the context of the present invention are provided herein.

For example, the probe may comprise porphyrin as an organic fluorescent core, wherein one or more of the sp²-carbon atoms are substituted with phosphonic acid or arsonic acid, wherein the P or As atom of the phosphonic acid or arsonic acid group are linked to the sp²-carbon atom. The phosphonic acid or arsonic acid group may be linked directly to the sp²-carbon of the porphyrin core, or via a suitable tether that extends the conjugated electron system of the core, such as a phenyl tether.

Preferred non-limiting examples of fluorescent probes of the invention, which can be used in a method of the present invention as disclosed herein are listed in Table 1 below.

In the displayed embodiments of Table 1 comprise at least one —PO₃H₂, —AsO₃H₂, —R^{II}PO₃H₂ or —R^{II}AsO₃H₂.

R^{I} can be a hydrogen atom or —PO₃H₂, —AsO₃H₂, —R^{II}PO₃H₂ and —R^{II}AsO₃H₂ and their monoesters. In embodiments, R^{I} can be a halogen atom (F, Cl, Br, I) or an aryl or alkyl group or a functional group known in organic chemistry. In preferred embodiments, R^{I} that are not —PO₃H₂, —AsO₃H₂, —R^{II}PO₃H₂ or —R^{II}AsO₃H₂ are H. In further preferred embodiments, R^{I} that are not —PO₃H₂, —AsO₃H₂, —R^{II}PO₃H₂ or —R^{II}AsO₃H₂ are alkyl, such as preferably methyl, ethyl or other short alkyls with 1-6 C-atoms. Different R^{I} can be the same or different within one of the disclosed formulas. The displayed embodiments of Table 1 comprise at least one —PO₃H₂, —AsO₃H₂, —R^{II}PO₃H₂ or —R^{II}AsO₃H₂. If no —PO₃H₂, —AsO₃H₂, —R^{II}PO₃H₂ or —R^{II}AsO₃H₂ is specifically displayed, at least one R^{I} is —PO₃H₂, —AsO₃H₂, —R^{II}PO₃H₂ or —R^{II}AsO₃H₂. In certain preferred embodiments, 2, 3, 4, 5, 6, 7 or 8 R^{I} are —PO₃H₂, —AsO₃H₂, —R^{II}PO₃H₂ or —R^{II}AsO₃H₂.

R^{II} is preferably a phenyl, biphenyl or triphenyl group or an aryl group, such as in the example phenylphosphonic acid. Preferably, different RH within one probe are the same. However, in embodiments, different RH within one molecule can also be different.

TABLE 1
Preferred non-limiting examples of fluorescent probes of the invention, which can be
used in methods of the invention as disclosed herein.

| | | |
|---|---|---|
| Structural formula of fluorescent probe | Name/Notes | Formula No. |

Fluorescent Phophonic acids with phthalocyanine core:

| | | |
|---|---|---|
| 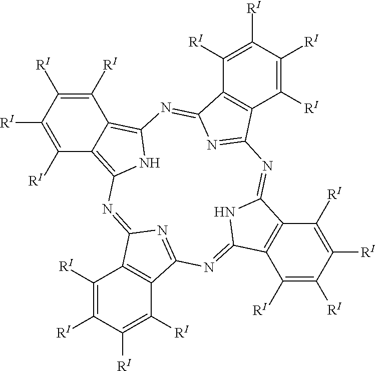 | | No. 1 |
| 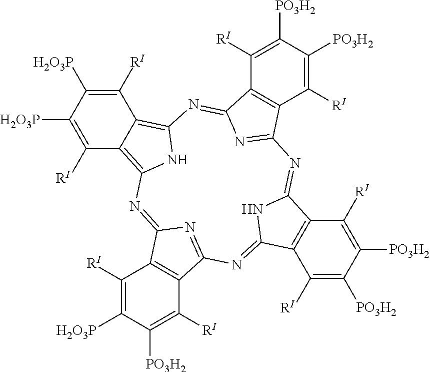 | Phthalocyanine hexaphosphonic acid | No. 2 |
| 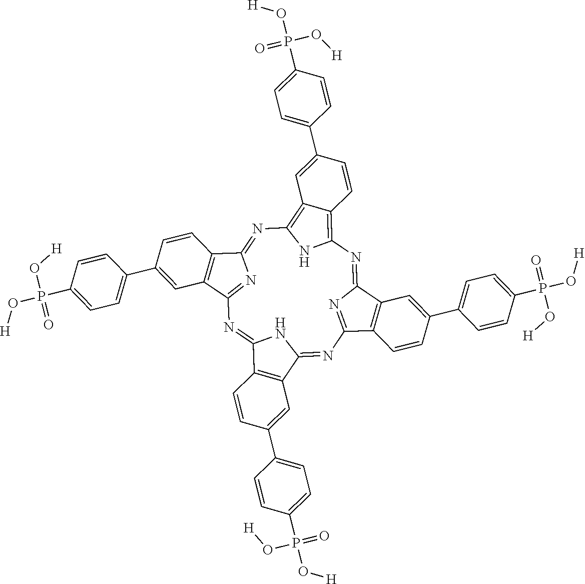 [SMILES: O=P(O)(O)c1ccc(-c2ccc3c(c2)-c2nc-3nc3[nH]c(nc4nc(nc5[nH]c(n2)c2ccc(-c6ccc(P(=O)(O)O)cc6)cc52)-c2ccc(-c5ccc(P(=O)(O)O)cc5)cc2-4)c2ccc(-c4ccc(P(=O)(O)O)cc4)cc32)cc1] | Tetra phenylphosphonic acid tethered phthalocyanine | No. 3 |

Fluorescent phosphonic acids with porphyrin core:

| | | |
|---|---|---|
| 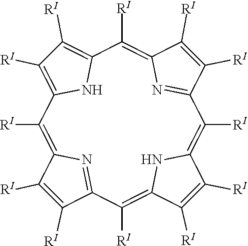 | | No. 4 |
| 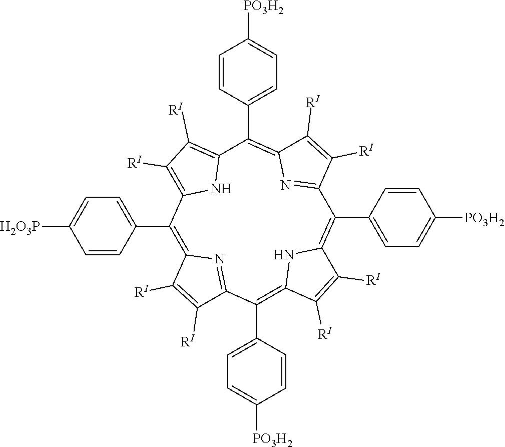 | Compound called p-H₈TPPA, when all R^{I} are H. | No. 5 |
| 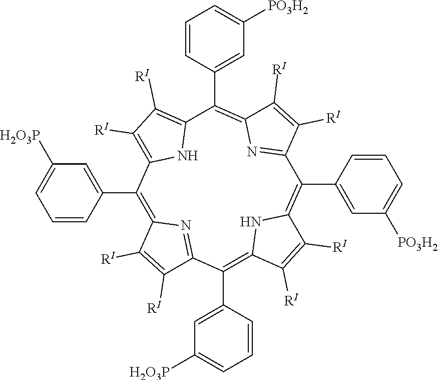 | Compound called m-H₈TPPA, when all R^{I} are H. | No. 6 |
| 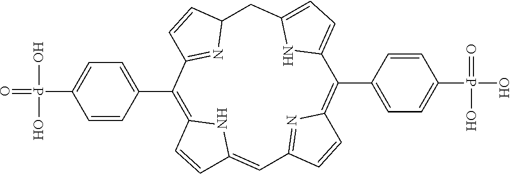 [SMILES: O=P(O)(O)c1ccc(/C2=C3\C=CC(=N3)/C=c3/cc/c([nH]3)=C(\c3ccc(P(=O)(O)O)cc3)C3=NC(C=C3)Cc3ccc2[nH]3)cc1] | 21H,23H-Porphine, 5, 15- bis(phenylphosphonic acid) | No. 7 |
| 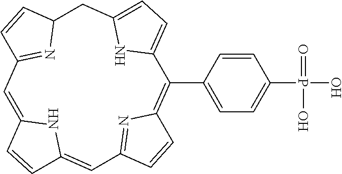 [SMILES: O=P(O)(O)c1ccc(/C2=C3\C=CC(=N3)/C=c3/cc/c([nH]3)=C/C3=NC(C=C3)Cc3ccc2[nH]3)cc1] | 21H,23H-Porphine, 5- phenylphosphonic acid | No. 8 |
| 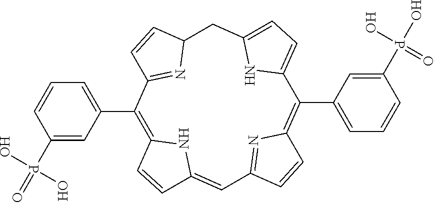 [SMILES: O=P(O)(O)c1cccc(/C2=C3\C=CC(=N3)/C=c3/cc/c([nH]3)=C(\c3cccc(P(=O)(O)O)c3)C3=NC(C=C3)Cc3ccc2[nH]3)c1] | 21H,23H-Porphine, 5, 15- bis(phenyl-3-phosphonic acid) | No. 9 |
| 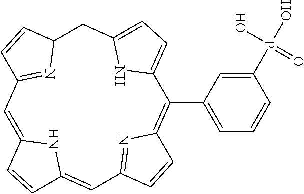 [SMILES: O=P(O)(O)c1cccc(/C2=C3\C=CC(=N3)/C=c3/cc/c([nH]3)=C/C3=NC(C=C3)Cc3ccc2[nH]3)c1] | 21H,23H-Porphine, 5-phenyl-3- phosphonic acid | No. 10 |
| 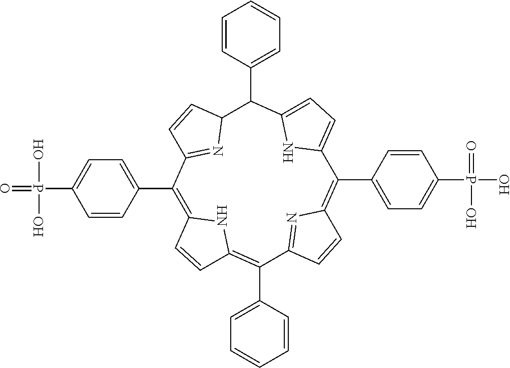 [SMILES: O=P(O)(O)c1ccc(/C2=C3\C=CC(=N3)/C(c3ccccc3)=c3/cc/c([nH]3)=C(\c3ccc(P(=O)(O)O)cc3)C3=NC(C=C3)C(c3ccccc3)c3ccc2[nH]3)cc1] | 21H,23H-Porphine, 5, 15- bis(phenyl-4-phosphonic acid), 10, 20-bis(phenyl) | No. 11 |
| 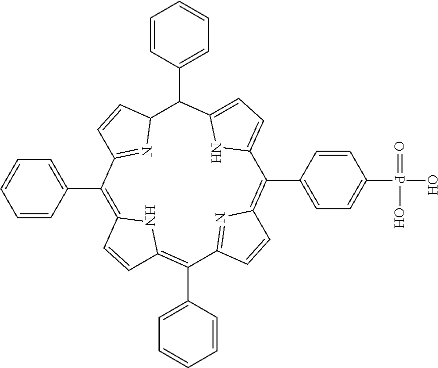 [SMILES: O=P(O)(O)c1ccc(/C2=C3\C=CC(=N3)/C(c3ccccc3)=c3/cc/c([nH]3)=C(\c3ccccc3)C3=NC(C=C3)C(c3ccccc3)c3ccc2[nH]3)cc1] | 21H,23H-Porphine, 5- phenylphosphonic acid, 10, 15, 20-tris(phenyl) | No. 12 |

Fluorescent phosphonic acids with coumarin core:

| | | |
|---|---|---|
| 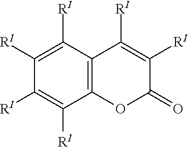 | | No. 13 |
| 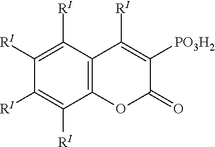 | In a preferred embodiment of No. 14, R^{I} are H. Coumarin-3-phosphonic acid | No. 14 |
| 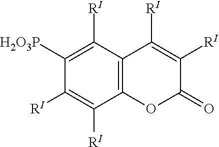 | Coumarin-6-phosphonic acid | No. 15 |

Fluorescent phosphonic acids with rhodamine core:

| | |
|---|---|
| 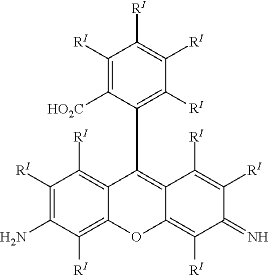 | No. 16 |

Fluorescent phosphonic acids with fluorescein core:

| | | |
|---|---|---|
| 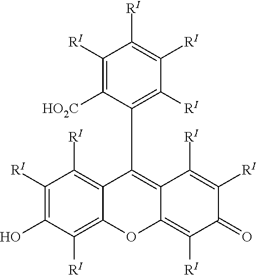 | | No. 17 |
| 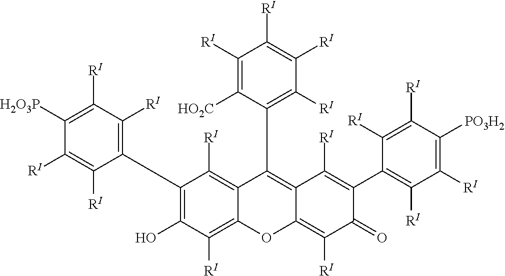 | Phenylphosphonic acid tethered fluorescein at the 2′ and 7′ positions 2′,7′- fluoresceindiphenylphosphonic acid | No. 18 |
| 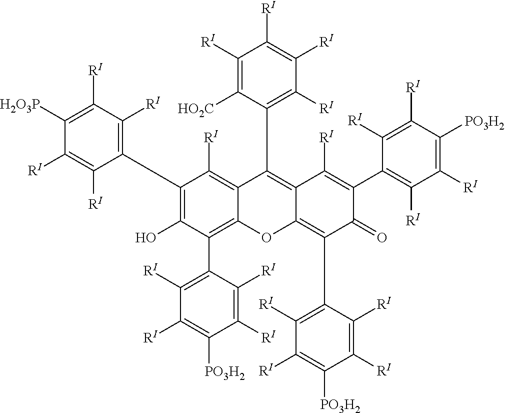 | Phenylphosphonic acid tethered fluorescein at the 2′,4′,5′,7′ positions 2′,4′,5′,7′- fluoresceintetraphenyl- phosphonic acid | No. 19 |

Fluorescent phosphonic acids with acridine core

| | | |
|---|---|---|
| 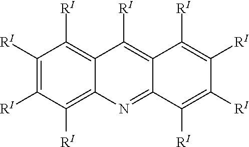 | | No. 20 |
| 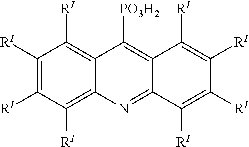 | Acridine-9-phosphonic acid | No. 21 |
| 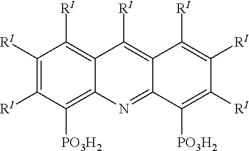 | 4,5-acridinedphosphonic acid | No. 22 |

Fluorescent phosphonic acids with xanthene core:

| | | |
|---|---|---|
| 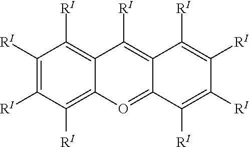 | | No. 23 |
| 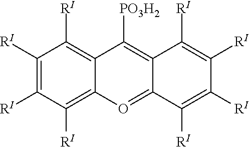 | Xanthene-9-phosphonic acid | No. 24 |
| 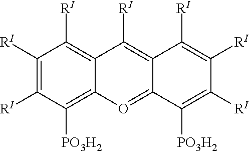 | 4,5-xanthenediphosphonic acid | No. 25 |

Fluorescent phosphonic acids with carbazole core:

| | | |
|---|---|---|
| 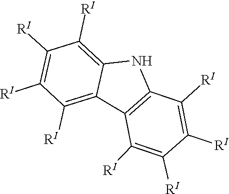 | | No. 26 |
| 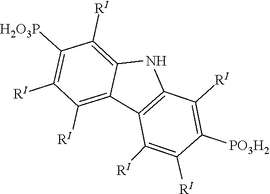 | 2,7-carbazolediphosphonic acid | No. 27 |

Further examples of probes for use in the method of the invention:

| | | |
|---|---|---|
| 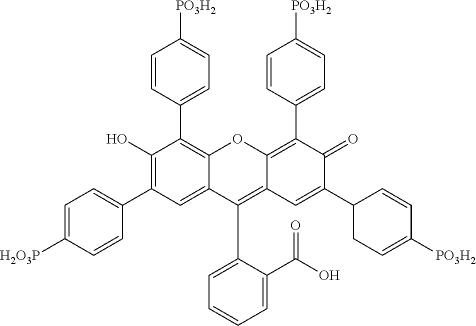 | | No. 28 |
| 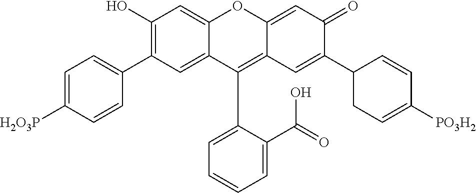 | | No. 29 |
| 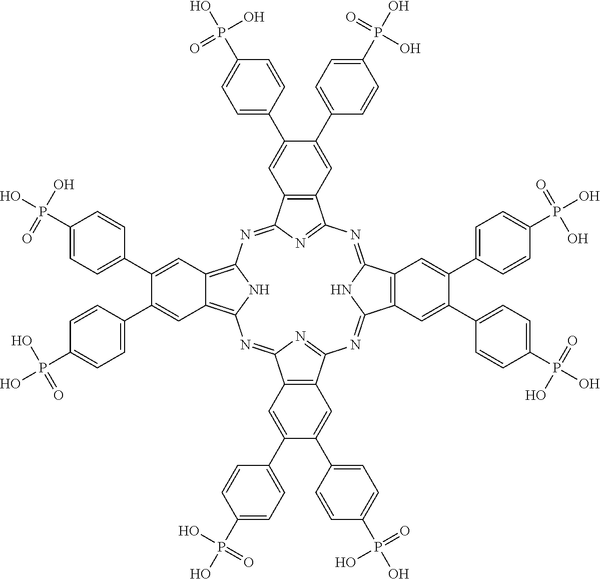 [SMILES: O=P(O)(O)c1ccc(-c2cc3c(cc2-c2ccc(P(=O)(O)O)cc2)-c2nc-3nc3[nH]c(nc4nc(nc5[nH]c(n2)c2cc(-c6ccc(P(=O)(O)O)cc6)c(-c6ccc(P(=O)(O)O)cc6)cc52)-c2cc(-c5ccc(P(=O)(O)O)cc5)c(-c5ccc(P(=O)(O)O)cc5)cc2-4)c2cc(-c4ccc(P(=O)(O)O)cc4)c(-c4ccc(P(=O)(O)O)cc4)cc32)cc1] | | No. 30 |
| 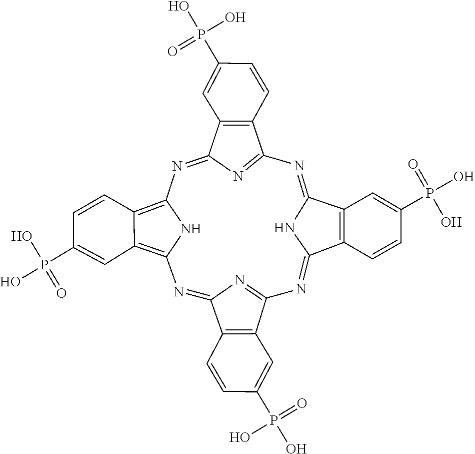 [SMILES: O=P(O)(O)c1ccc2c(c1)-c1nc-2nc2[nH]c(nc3nc(nc4[nH]c(n1)c1ccc(P(=O)(O)O)cc41)-c1ccc(P(=O)(O)O)cc1-3)c1ccc(P(=O)(O)O)cc21] | | No. 31 |
| 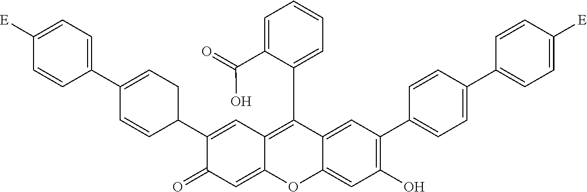 | E = -PO₃H₂, -AsO₃H₂, | No. 32 |
| 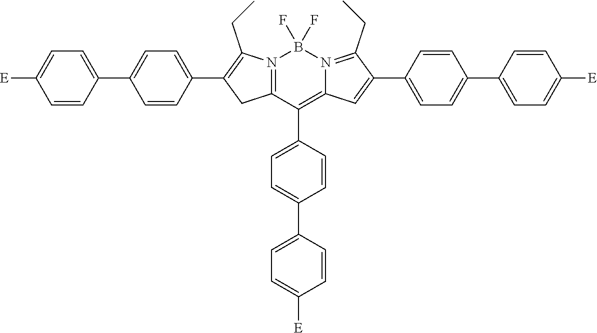 | E = -PO₃H₂, -AsO₃H₂, | No. 33 |
| 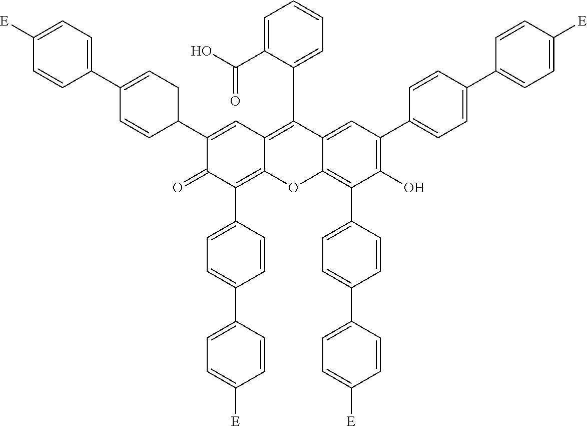 | E = -PO₃H₂, -AsO₃H₂, | No. 34 |
| 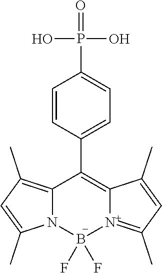 | | No. 35 |
| 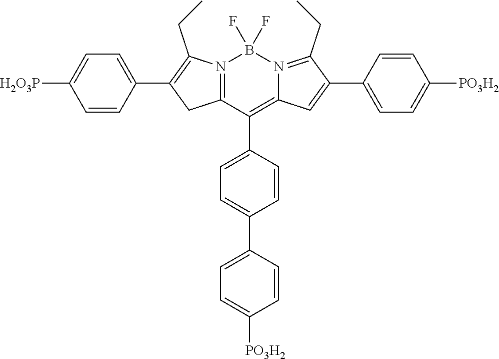 | | No. 36 |
| 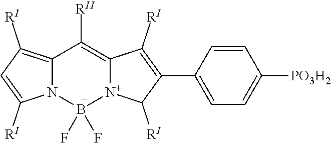 | | No. 37 |
| 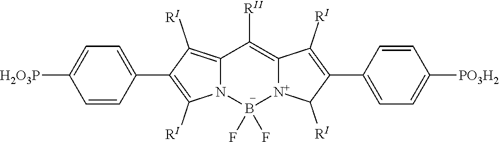 | | No. 38 |

It was found that presence of calcium and calcium depositions in tissue and binding of such depositions by the metal binding functional groups of the probes of the invention leads to changes of fluorescence of the probes and also enables specific detection of such depositions in a tissue or tissue sample. In particular, binding of a probe of the invention to a calcium deposition in a tissue sample or tissue leads to an increase in fluorescence, such as an increase in emission (of light). The detection of the calcium deposition is facilitated by parallel analysis of a negative control sample, which can be sample known to not comprise a calcification, or a sample that is treated under conditions that do not enable binding of the probe.

It is preferred that the probe to be used in the context of the present invention is cell permeable, so it can traverse the cell membrane and bind to calcium and/or depositions present inside a biological cell, for example in the cytoplasm.

Cell permeable probes are probes that efficiently cross the cell membrane and access the cytosol of a cell. Molecules that can readily cross cell membranes are frequently needed in biological research and medicine and besides fluorescent probes functioning as metal ion indicators, as the probes of the present invention, cell permeability is also important for pH indicators, fluorescent dyes, crosslinking molecules, fluorogenic enzyme substrates, and various protein inhibitors that may be functioning as pharmacological drugs. Due to the extensive research in this field, it is known to a skilled person how to design or modify the probes of the invention to achieve cell permeability. For example, it is known to the skilled person that amphipathic molecules are likely to be cell permeable. In embodiments, charged groups can be chemically masked for enabling cell permeability, followed by enzymatic removal of the masking group (which can be, for example, an acetoxymethyl ester or an ethyl ester) once the probe is inside the cell.

A cell comprised by a bodily sample or bodily tissue of the invention that is exposed to a cell permeable fluorescent probe as described herein may be analyzed by microscopic analysis, for example by using a confocal fluorescent microscope, to measure fluorescence.

In fluorescence microscopy, fluorescence is used to study the properties of organic or inorganic substances, and in particular of biological cells. A fluorescence microscope is a microscope that uses fluorescence to generate an image, whether it is a more simple set up like an epifluorescence microscope or a more complicated design such as a confocal microscope, which uses optical sectioning to get better resolution of the fluorescence image.

The basic principle of fluorescent microscopy is that a specimen is illuminated with light of a specific wavelength (or wavelengths), which is absorbed by the fluorophores comprised by the specimen (or sample), causing them to emit light of longer wavelengths (i.e., of a different color than the absorbed light). The illumination light is separated from the much weaker emitted fluorescence using a spectral emission filter. Typical components of a fluorescence microscope are a light source (xenon arc lamp or mercury-vapor lamp are common; more advanced forms are high-power LEDs and lasers), the excitation filter, the dichroic mirror (or dichroic beam splitter), and the emission filter (see figure below). The filters and the dichroic beam splitter are chosen to match the spectral excitation and emission characteristics of the fluorophore used to label the specimen. In this manner, the distribution of a single fluorophore (color) is imaged at a time. Multi-color images of several types of fluorophores must be composed by combining several single-color images.

In preferred embodiments of the invention, cells comprised by a liquid sample are analyzed by confocal microscopy. Confocal microscopy is most frequently performed as confocal laser scanning microscopy (CLSM) or laser confocal scanning microscopy (LCSM) and is an optical imaging technique for increasing optical resolution and contrast of a micrograph or a microscopy sample by means of using a spatial pinhole to block out-of-focus light in image formation. The analyzed sample may comprise a fixed or a living cell or tissue sample. Capturing multiple two-dimensional images at different depths in a sample enables the reconstruction of three-dimensional structures (a process known as optical sectioning) within an object. Confocal microscopy enables easy quantification of the acquired fluorescence data.

Light travels through the sample under a conventional fluorescent microscope as far into the specimen as it can penetrate, while a confocal microscope only focuses a smaller beam of light at one narrow depth level at a time. The CLSM achieves a controlled and highly limited depth of focus.

Furthermore, the probes of the invention can be used in the context of non-invasive fluorescence imaging methods, which have the potential to provide in vivo diagnostic information for many clinical specialties. Techniques have been developed over the years for simple ocular observations following UV excitation to sophisticated spectroscopic imaging using advanced equipment. NIR radiation is also frequently used in fluorescence based diagnostic methods.

In embodiments, the detecting and preferably quantifying a signal from the fluorescent probe of the invention and thereby preferably detecting calcium or a calcification requires detection and optionally quantification of the measured fluorescent signal. Such detection and/or quantification may require comparison of the measured signal to a reference sample, which may be a sample known not to comprise calcium and/or calcifications, and/or samples of known calcium concentration. Such reference samples may also be referred to as calibration samples. In this context, the term calibration relates to the comparison of measured fluorescent signal generated from a sample of interest with the fluorescent signal of a calibration standard of known metal ion concentration. In the context of a diagnostic method, a reference sample can be a different subject/patient, for example a subject known to not comprise a calcification in a respective tissue, of a subject that was not administered the probe of the invention.

The fluorescent probe of the invention can be use for detecting calcium and/or a calcification, for example in the context of a diagnostic or prognostic method.

Calcium is a chemical element with the symbol Ca and atomic number 20. As an alkaline earth metal, calcium is a reactive metal that forms a dark oxide-nitride layer when exposed to air. Calcium and in particular divalent calcium ions present in calcium salts were surprisingly found to be bound by the probes of the invention and to lead to an increase in fluorescence emission by the probes upon calcium binding.

The probes of the invention in preferably bind to calcifications. As used herein, a calcification (also referred to as a calcium deposition) is the accumulation of calcium salts in a body tissue. It normally occurs in the formation of bone, but calcium can be deposited abnormally in soft tissue, causing it to harden in some cases. Calcifications may be classified on whether there is mineral balance or not, and the location of the calcification. Calcification may also refer to the processes of normal mineral deposition in biological systems, such as the formation of bones.

Calcification of soft tissue (arteries, cartilage, heart valves, etc.) can be caused by vitamin K2 deficiency or by poor calcium absorption due to a high calcium/vitamin D ratio. This can occur with or without a mineral imbalance. Intake of excessive vitamin D can cause vitamin D poisoning and excessive intake of calcium from the intestine, when accompanied by a deficiency of vitamin K (perhaps induced by an anticoagulant) can result in calcification of arteries and other soft tissue. Such metastatic soft tissue calcification is mainly in tissues containing “calcium catchers” such as elastic fibres or sour mucopolysaccharides. These tissues especially include the lungs (pumice lung) and the aorta.

Dystrophic calcification, without a systemic mineral imbalance, whereas so-called metastatic calcification relates to a systemic elevation of calcium levels in the blood and all tissues.

Calcification can be pathological or a standard part of the aging process. Nearly all adults show calcification of the pineal gland. Potential locations of calcification in a body, preferably a mammalian or human body, include extraskeletal calcification, e.g. calciphylaxis; Cacifications in the brain, e.g. primary familial brain calcification (Fahr's syndrome); calcification of the choroid plexus usually in the lateral ventricles; tumor calcification; arthritic bone spurs; kidney stones; gall stones; heterotopic bone; tonsil stones; intra-ocular calcification; calcified nodules in the retina occurring in age-related macular degeneration; and skin calcifications.

The invention can be used to detect such calcification in any bodily tissue, either after administration to a subject suspected of having a calcification of a respective tissue or suffering from a certain disease associated with calcifications, or in vitro in an isolated sample provided by a subject. Accordingly, the probe of the invention can be used in a diagnostic or prognostic method for determining the presence of the risk of a future development of a disease associated with tissue calcifications. Furthermore, the probes of the invention can be used to detect and measure teeth, bone and cartilage formation and/or regeneration in various context, such as after injury, or over the course of a regeneration therapy.

In embodiments, the probe of the invention can be used in the context of the methods and processes disclosed herein for the detection and optionally imaging of hydroxyapatite (HAP). HAP, also called hydroxylapatite or HA, is a naturally occurring mineral form of calcium apatite with the formula Ca₅(PO₄)₃(OH), but it is usually written Ca₁₀(PO₄)₆(OH)₂ to denote that the crystal unit cell comprises two entities. Hydroxyapatite is the hydroxyl endmember of the complex apatite group. The OH— ion can be replaced by fluoride, chloride or carbonate, producing fluorapatite or chlorapatite. It crystallizes in the hexagonal crystal system. Pure hydroxyapatite powder is white. Naturally occurring apatites can, however, also have brown, yellow, or green colorations, comparable to the discolorations of dental fluorosis. Up to 50% by volume and 70% by weight of human bone is a modified form of hydroxyapatite, known as bone mineral. Carbonated calcium-deficient hydroxyapatite is the main mineral of which dental enamel and dentin are composed. Hydroxyapatite crystals are also found in the small calcifications, within the pineal gland and other structures, known as corpora arenacea or ‘brain sand’.

Hydroxyapatite is present in bone and teeth; bone is made primarily of HA crystals interspersed in a collagen matrix. 65 to 70% of the mass of bone is HA. Similarly, HA is 70 to 80% of the mass of dentin and enamel in teeth. In enamel, the matrix for HA is formed by amelogenins and enamelins instead of collagen. Hydroxyapatite deposits in tendons around joints results in the medical condition calcific tendinitis.

Hydroxyapatite is added to some variations of cornstarch based baby powder to help moisturize and soften skin. In a medical context HA is increasingly used to make bone grafting materials as well as dental prosthetics and repair. Some implants, e.g. hip replacements, dental implants and bone conduction implants, are coated with HA. As the native dissolution rate of hydroxyapatite in-vivo, around 10 wt % per year, is significantly lower than the growth rate of newly formed bone tissue, in its use as a bone replacement material, ways are being sought to enhance its solubility rate and thus promote better bioactivity. Hydroxyapatite is added to special toothpastes as an additive to prevent tooth decay and to counteract tooth sensitivity.

The probes of the invention can be used in any kind of application requiring the imaging or detection of calcification of HAP. Besides imaging of HAP or calcification in bodily sample or in the body, the probes can be used for determining the presence or calcium, in particular calcifications and HAP in a sample, for example a sample of a liquid or gel or powder, such as the products described herein.

Calcifications in body tissues are currently detected using various techniques, such as computed tomography scans, ultrasound, cone beam CT and other medical diagnostic imaging techniques known in the art (see for example Saade C, Najem E, Asmar K, Salman R, El Achkar B, Naffaa L. “Intracranial calcifications on CT: an updated review”. J Radiol Case Rep. 2019 Aug. 31; 13(8):1-18; Baldwin P. “Breast calcification imaging.” Radiol Technol. 2013 March-April; 84(4):383M-404M; “Calcific tendinitis of the rotator cuff: state of the art in diagnosis and treatment.” Merolla G, Singh S, Paladini P, Porcellini G. J Orthop Traumatol. 2016 March; 17(1):7-14; “Calcific aortic valve stenosis: hard disease in the heart: A biomolecular approach towards diagnosis and treatment.” Peeters F E C M, Meex S J R, Dweck M R, Aikawa E, Crijns H J G M, Schurgers L J, Kietselaer B L J H. Eur Heart J. 2018 Jul. 21; 39(28):2618-2624).

Bodily tissue that can contain calcifications that can be imaged or analyzed using the probes of the invention include soft tissues, such as brain, eye, kidney, skin, gastrointestinal organs, liver, organs, tendons, ligaments, fascia, skin, fibrous tissues, fat, synovial membranes, muscles, nerves or blood vessels; bones; teeth; cartilage; joints.

The probes of the invention can be used for detecting bone growth and/or resorption.

The probes of the invention can be used for detecting breast calcification and in particular microcalcifications in breast tissue. Breast calcifications are small dots of calcium salts that can occur anywhere in the breast tissue. They are very small so you won't be able to feel them, and they don't cause any pain. Breast calcifications are very common. They are usually due to benign (not cancer) changes that occur as part of aging. Breast calcifications are common on mammograms, and they're especially prevalent after age 50. Although breast calcifications are usually noncancerous (benign), certain patterns of calcifications—such as tight clusters with irregular shapes and fine appearance—may indicate breast cancer or precancerous changes to breast tissue. On a mammogram, breast calcifications can appear as macrocalcifications or microcalcifications: Macrocalcifications show up as large white dots or dashes. They're almost always noncancerous and require no further testing or follow-up. Microcalcifications show up as fine, white specks, similar to grains of salt. They're usually noncancerous, but certain patterns can be an early sign of cancer.

The probes of the invention can be used for detecting drusen formation to detect early stages of age-related macular degeneration disease. Drusen are small yellow or white accumulations of extracellular material that build up between Bruch's membrane and the retinal pigment epithelium of the eye. The presence of a few small (“hard”) drusen is normal with advancing age, and most people over 40 have some hard drusen. However, the presence of larger and more numerous drusen in the macula is a common early sign of age-related macular degeneration (AMD).

In the context of the methods of the invention and uses of the probes of the invention, the probe may be brought into contact with a tissue or tissue sample to be analyzed for the presence of a calcification. The tissue or sample can also be or comprise a lumen or a cell or multiple cells, which can preferably be imaged and analyzed by using the fluorescent probes of the invention that specifically bind to calcifications and enhance their fluorescence emission upon binding. Accordingly, the invention provides means for specifically detecting and imaging tissue calcifications by using the probes, which are highly advantageous due to the specific binding, easy detection and enhance signal emission in positive samples. These properties of the probes make it possible to easily distinguish positive detection events from background signals and make highly sensitive and specific identification of calcifications possible.

The invention further relates to a contrast agent, comprising a fluorescent probe of the invention. A contrast agent may also be termed “imaging agent” in the context of the invention. A contrast agent (or contrast medium) is a substance used to increase the contrast of structures or fluids within the body in medical imaging. Contrast agents can absorb or alter external electromagnetism or ultrasound, which is different from radiopharmaceuticals, which emit radiation themselves. In x-rays, contrast agents enhance the radiodensity in a target tissue or structure. In MRIs, contrast agents shorten (or in some instances increase) the relaxation times of nuclei within body tissues in order to alter the contrast in the image. Contrast agents are commonly used to improve the visibility of blood vessels and the gastrointestinal tract, but suitable and specific contrast can be used for imaging of specific structures in a body, depending on their binding properties. Accordingly, the probes of the invention can be used in various imaging methods as a contrast agent for imaging calcium and calcifications. The probes of the invention may also be used in the context of NRI imaging, for example as a contrast agent. The deep tissue propagation of near-infrared (NIR) light between 700-900 nm offers opportunities for diagnostic imaging when employing sensitive detection techniques and NIR excitable fluorescent agents that target and report disease and metabolism. NRI and suitable fluorescent contrast agents can be used for illuminating tissues and monitoring the re-emitted fluorescence for tomographic reconstruction, for example in fluorescence enhanced NIR optical imaging.

In embodiments, the invention relates to a method for diagnosis, prognosis, risk assessment, monitoring, therapy guidance and/or therapy control of a medical/clinical condition of a subject associated with calcium depositions in a bodily sample or tissue.

The term “clinical diagnostics” or “diagnosis” relates to the recognition and (early) detection of a clinical condition of a subject. “Prognosis” relates to the prediction of an outcome or a specific risk for a subject. This may also include an estimation of the chance of recovery or the chance of an adverse outcome for said subject. In the present invention, the terms “risk assessment” and “risk stratification” relate to the grouping of subjects into different risk groups according to their further prognosis. Risk assessment also relates to stratification for applying preventive and/or therapeutic measures. The term “therapy stratification” in particular relates to grouping or classifying patients into different groups, such as risk groups or therapy groups that receive certain differential therapeutic measures depending on their classification. “Monitoring” relates to keeping track of an already diagnosed condition, disorder, complication or risk, e.g., to analyze the progression of the disease or the influence of a particular treatment or therapy on the disease progression of the disease in a patient. The term “therapy monitoring” or “therapy control” in the context of the present invention refers to the monitoring and/or adjustment of a therapeutic treatment of said subject, for example by obtaining feedback on the efficacy of the therapy. As used herein, the term “therapy guidance” refers to application of certain therapies, therapeutic actions or medical interventions based on the value/level of one or more biomarkers and/or clinical parameter and/or clinical scores, in particular the presence and concentration of metal ions in a patient sample. This includes the adjustment of a therapy or the discontinuation of a therapy.

In embodiments, the method of the invention is used in clinical diagnostics for determining available levels of free metal ions as a diagnostic marker for nutrient supply.

The instant disclosure also includes kits, packages and multi-container units containing the herein described reagents, such as the fluorescent probes and potentially buffer, chelators and/or other useful reagents for carrying out the method of the invention, and the use of such kits for performing the inventive method.

### FIGURES

The invention is further described by the following figures. These are not intended to limit the scope of the invention but represent preferred embodiments of aspects of the invention provided for greater illustration of the invention described herein.

### DESCRIPTION OF THE FIGURES

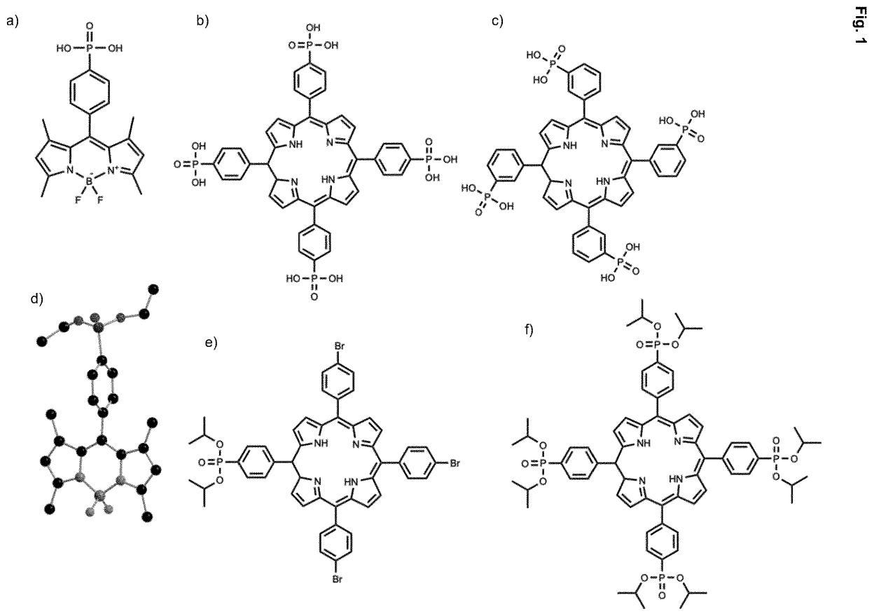

FIG. 1: The fluorescent probes used in this study a) BODIPY-PPA b) p-H₈TPPA c) m-H₈TPPA d) crystal structure of BODIPY-PPA-2Et₂ e) p-TBr3PPA-iPr₂ f) p-H₈TPPA-iPr₈.

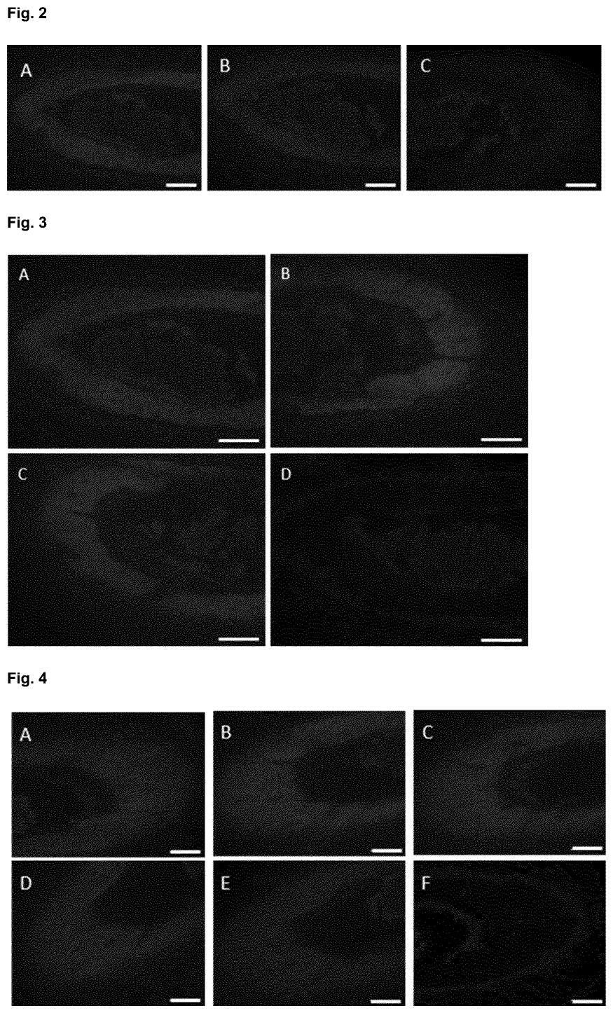

FIG. 2: Mouse ribs incubated with p-H₈TPPA (λ_{ex/em} 595/613 nm; exposure time—200 ms) diluted to 1 mg/mL (A), 0.1 mg/mL (B) and 0.01 mg/mL (C) and counter-stained with DAPI (λ_{ex/em} 359/461 nm; exposure time—200 ms); scalebar=100 μm (microscope, objective, filters or cubes, camera, or are details elsewhere).

FIG. 3: Mouse ribs incubated with p-H₈TPPA (λ_{ex/em} 595/613 nm; exposure time—200 ms) at 1 mg/mL in HEPES (A), PBS (B), TBS (C) and dH₂O (D) and counter-stained with DAPI (λ_{ex/em} 359/461 nm; exposure time—200 ms); scalebar=100 μm.

FIG. 4: Mouse ribs incubated with p-H₈TPPA (λ_{ex/em} 595/613 nm; exposure time—200 ms) at 1 mg/mL for 120, 60, 30, 20 and 10 mins (A-E, respectively) in HEPES buffer and counter-stained with DAPI (λ_{ex/em} 359/461 nm; exposure time—200 ms). A negative control of mouse ribs incubated with HEPES buffer alone is also included (F). Scalebar=100 μm.

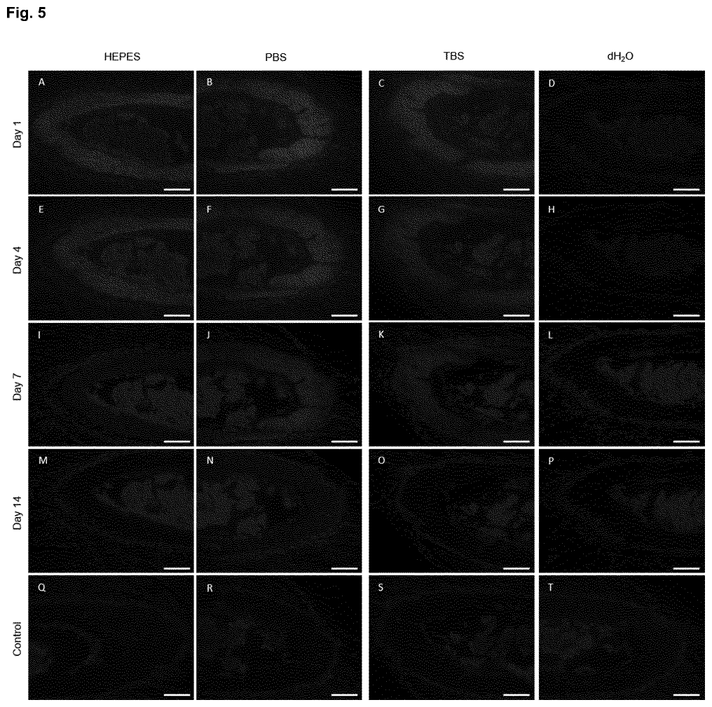

FIG. 5: Mouse ribs incubated with p-H₈TPPA (λ_{ex/em} 595/613 nm; exposure time—200 ms) diluted to 1 mg/mL in HEPES, PBS, TBS and dH₂O (A-D, respectively) and counter-stained with DAPI (λ_{ex/em} 359/461 nm; exposure time—200 ms). Images were captured on the day of staining (A-D), as well as after 4 days (E-H), 7 days (I-L) and 14 days (M-P). A negative control of mouse ribs incubated with each buffer alone is also included (Q-T, respectively). Scalebar=100 μm.

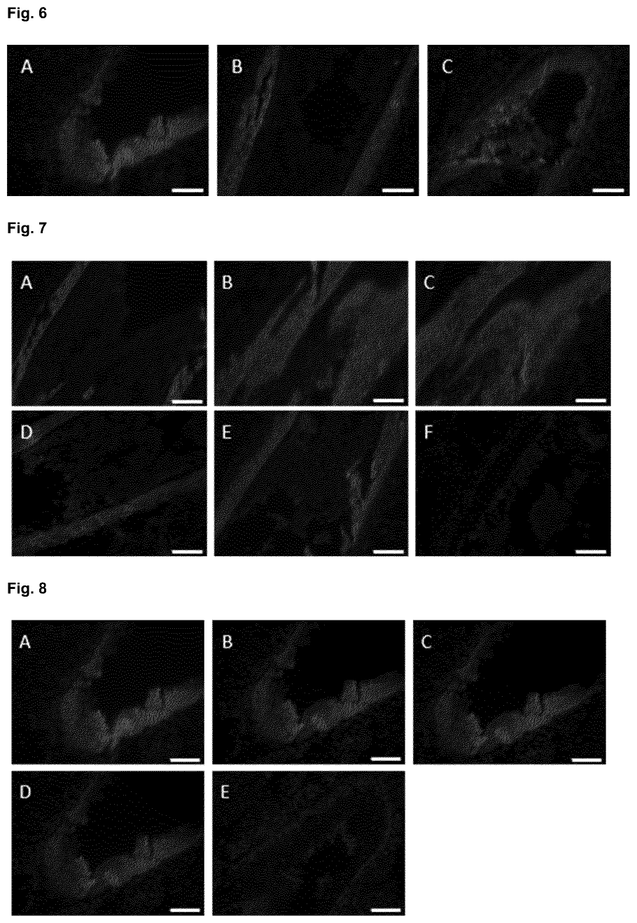

FIG. 6: Mouse ribs incubated with m-H₈TPPA (λ_{ex/em} 578/603 nm; exposure time—100 ms) diluted to 1 mg/mL (A), 0.1 mg/mL (B) and 0.01 mg/mL (C) and counter-stained with DAPI (λ_{ex/em} 359/461 nm; exposure time—100 ms); scalebar=100 μm.

FIG. 7: Mouse ribs incubated with m-H₈TPPA (λ_{ex/em} 578/603 nm; exposure time—100 ms) at 1 mg/mL for 120, 60, 30, 20 and 10 mins (A-E, respectively) in HEPES buffer and counter-stained with DAPI (λ_{ex/em} 359/461 nm; exposure time—100 ms). A negative control of mouse ribs incubated with HEPES buffer alone is also included (F). Scalebar=100 μm.

FIG. 8: Mouse ribs incubated with m-H₈TPPA (λ_{ex/em} 578/603 nm; exposure time—100 ms) at 1 mg/mL for 120 mins in HEPES and imaged at day 1 (A), day 4 (B), day 7 (C) and day 14 (D) and counter-stained with DAPI (λ_{ex/em} 359/461 nm; exposure time—100 ms). A negative control of mouse ribs incubated with HEPES buffer alone is also included (E). Scalebar=100 μm.

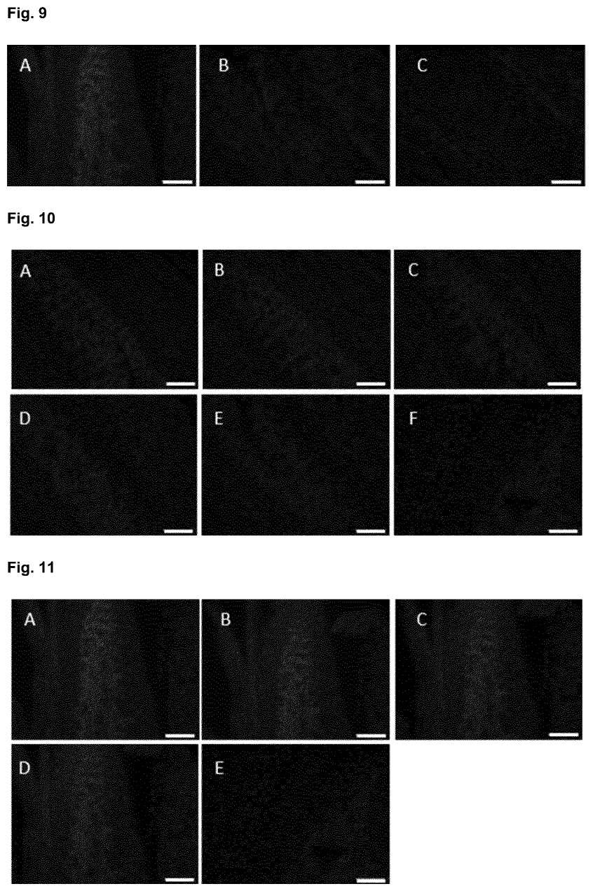

FIG. 9: Mouse ribs incubated with BODIPY-PPA (λ_{ex/em} 578/603 nm; exposure time—400 ms) diluted to 1 mg/mL (A), 0.1 mg/mL (B) and 0.01 mg/mL (C) in HEPES and counter-stained with DAPI (λ_{ex/em} 359/461 nm; exposure time—100 ms); scalebar=100 μm.

FIG. 10: Mouse ribs incubated BODIPY-PPA (λ_{ex/em} 578/603 nm; exposure time—400 ms) at 1 mg/mL buffer for 120, 60, 30, 20 and 10 mins (A-E, respectively) in HEPES buffer and counter-stained with DAPI (λ_{ex/em} 359/461 nm; exposure time—100 ms). A negative control of mouse ribs incubated with HEPES buffer alone is also included (F). Scalebar=100 μm.

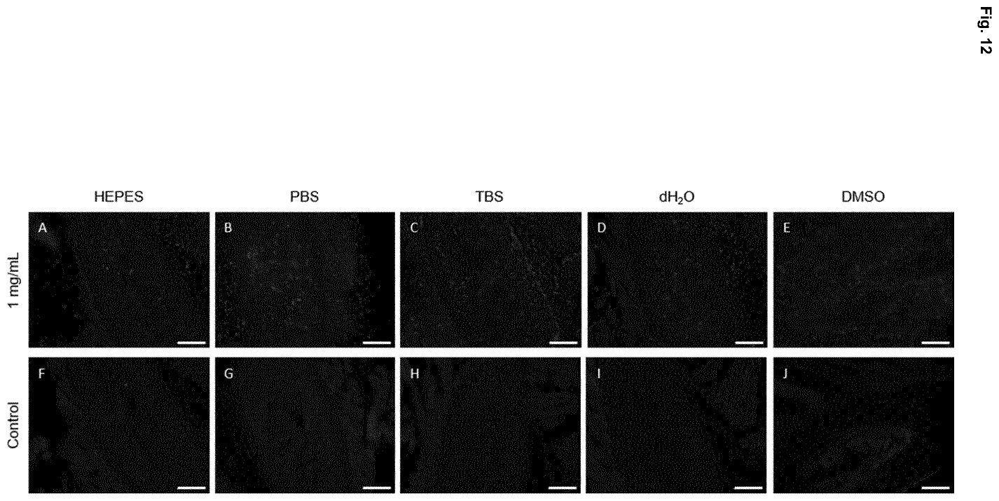

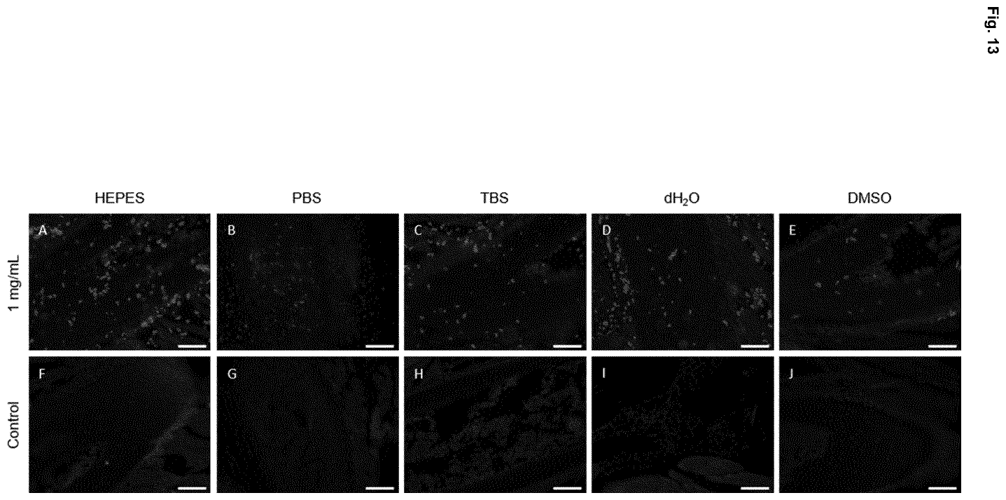

FIG. 11: Mouse ribs incubated with BODIPY-PPA (λ_{ex/em} 578/603 nm; exposure time—400 ms) at 1 mg/mL for 120 mins in HEPES and imaged at day 1 (A), day 4 (B), day 7 (C) and day 14 (D) and counter-stained with DAPI (λ_{ex/em} 359/461 nm; exposure time—100 ms). A negative control of mouse ribs incubated with HEPES buffer alone is also included (E). Scalebar=100 μm.

FIG. 12: Mouse ribs incubated with p-TBr3PPA-iPr₂ (λ_{ex/em} 595/613 nm; exposure time—400 ms) at 1 mg/mL in HEPES, PBS, TBS, dH₂O and DMSO (A-E, respectively) and counter-stained with DAPI (λ_{ex/em} 359/461 nm; exposure time—100 ms). A negative control of mouse ribs incubated with each buffer alone is also included (F-J, respectively). Scalebar=100 μm.

FIG. 13: Mouse ribs incubated with p-H₈TPPA-iPr₈ (λ_{ex/em} 595/613 nm; exposure time—400 ms) at 1 mg/mL in HEPES, PBS, TBS, dH₂O and DMSO (A-E, respectively) and counter-stained with DAPI (λ_{ex/em} 359/461 nm; exposure time—100 ms). A negative control of mouse ribs incubated with each buffer alone is also included (F-J, respectively). Scalebar=100 μm.

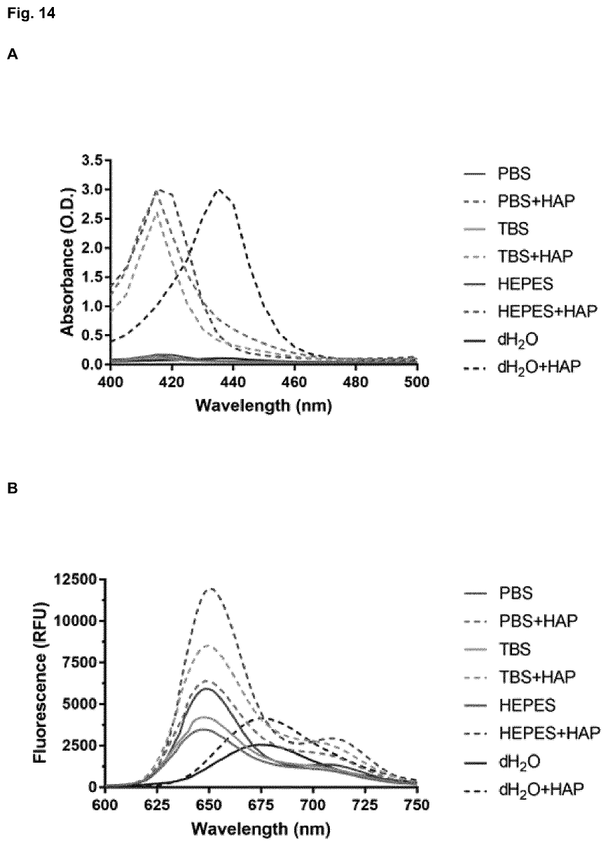

FIG. 14: FIG. 13 shows the fluorescent intensity increase of p-H₈TPPA upon hydroxyapatite (HAP) binding in comparison with the fluorescent intensity in the absence of HAP. The absorbance (A) and fluorescence (B) spectra of 0.01 mg mL⁻¹p-H₈TPPA in the presence (dashed lines) and absence (solid lines) of HAP. The dye was diluted to 0.01 mg mL⁻¹ in PBS (pH 7.4, green), TBS (pH 7.4, orange), HEPES (pH 7.4, purple) and d H₂O with absorbance and fluorescence spectra obtained using the Flexstation 3 microplate reader.

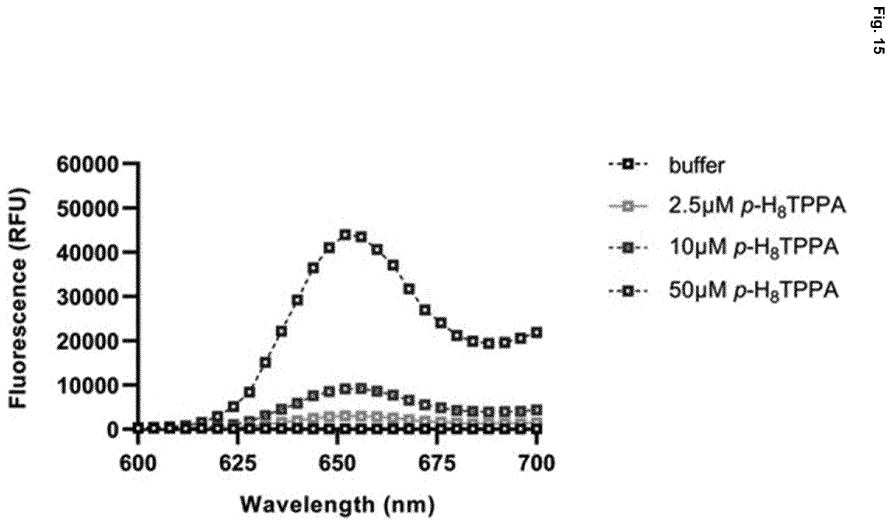

FIG. 15: Fluorescence spectra of THP-1 monocytes cells incubated with p-H₈TPPA.

### EXAMPLES

The invention is further described by the following examples. These are not intended to limit the scope of the invention but represent preferred embodiments of aspects of the invention provided for greater illustration of the invention described herein.

### SUMMARY OF THE EXAMPLES

Recent studies revealed the presence of microscopic particles of bone mineral within deposits in the retina (the best known are called drusen), and they seem to be associated with the development of these deposits in AMD, and in the retinal periphery, with Alzheimer Disease. We have synthesized a group of chemical probes for use in studying the deposition of the bone mineral by microscopy and other means. Using these novel methods, we seek to understand the biology of deposit formation and growth and use this knowledge to develop new diagnostics and potential treatments for these diseases.

Recently, it has emerged that mineralization in the retina with hydroxyapatite (HAP) and other species is correlated with the development of AMD and Alzheimer disease. While we have used legacy stains including classic tetracycline antibiotics as selective fluorescent stains for HAP in the retina, these compounds have some limitations and novel HAP-specific fluorescent stains may offer useful new properties including fluorescence imaging contrast using new mechanisms. These properties may further elucidate the HAP deposition process mechanism(s) and suggest novel treatments. Herein, the following compounds of the invention are used:
((4-(5,5-difluoro-1,3,7,9-tetramethyl-5H-414,514-dipyrrolo[1,2-c:2′,1′-f][1,3,2]diazaborinin-10-yl)phenyl)phosphonic acid) (BODIPY-PPA)
5,10,15,20-tetrakis[p-phenylphosphonic acid] porphyrin (p-H₈TPPA)
5,10,15,20-tetrakis[m-phenylphosphonic acid] porphyrin (m-H₈TPPA)

Characterization of Probes: The structure and purity of the probes were characterized by liquid chromatography, infrared, NMR, and/or mass spectrometry. Their optical properties were characterized by absorption and fluorescence spectrophotometry in the presence and absence of authentic hydroxyapatite, whitlockite, and other doped apatites. The dyes were also tested on mouse ribs as an example of hydroxyapatite in tissue.

We found that several of the synthesized probes bound HAP tightly, in some cases with alterations of their fluorescence properties, which we attribute to synergistic interactions arising from conjugation of the sp2 carbons of the aryl fluorophore moiety with the phosphonate moiety. Staining a cross section of mouse rib with meso-tetra(4-phosphorylphenyl)porphyrin (red) and DAPI (blue), together with autofluorescence (green) is depicted in the figure (A), with a DAPI-stained control section from the same mouse (B). Based on these preliminary results, staining appears specific for hydroxyapatite.

We conclude that these new fluorescent labels offer novel features that will be of use in elucidating the biology of retinal mineralization and its relationship to macular and peripheral deposit formation seen in AMD and Alzheimer disease, respectively.

### METHODS OF THE EXAMPLES

Mouse ribs (C57BL/6) were fixed in 4% paraformaldehyde (PFA). Ribs were processed for wax embedding through sequential immersion in increasing concentrations of ethanol followed by immersion in xylene/clearene and paraffin wax embedding. Rib sections (6 μm thickness) were dewaxed with xylene and rehydrated with decreasing concentrations of ethanol and distilled water (dH₂O). Rib sections were incubated with a variety of either namely((4-(5,5-difluoro-1,3,7,9-tetramethyl-5H-414,514-dipyrrolo[1,2-c:2′,1′-f][1,3,2]diazaborinin-10-yl)phenyl)phosphonic acid) (BODIPY-PPA), 5,10,15,20-tetrakis[p-phenylphosphonicacid] porphyrin (p-H₈TPPA) and 5,10,15,20-tetrakis[m-phenylphosphonic acid] porphyrin (m-H₈TPPA) dyes. These are 5,10,15,20-tetrakis[p-phenylphosphonic acid] porphyrin (p-H₈TPPA), as well as its diester form (p-H₈TPPA-iPr₈); 5,10,15-tris[p-bromophenyl]-20-[p-phenyl-, bis(1-methylethyl) ester phosphonic acid] porphyrin (p-TBr₃PPA-iPr₂) with bulkyisopropyl groups, and BODIPY-PPA-2Et₂ with two ethyl groups. p-H₈TPPA was incubated for 120 mins at 1 mg/mL in 2-[4-(2-hydroxyethyl)piperazin-1-yl]ethanesulfonic acid (HEPES, pH 7.4), phosphate-buffered saline (PBS, pH 7.4), tris-buffered saline (TBS, 7.4; Trizma base) and dH₂O. Meso-tetra(4-phosphorylphenyl)porphine, porph-tetra-meta-phenyl phosphonate, BODIPY-PPA; and BODIPY-PPA-2Et₂ were all incubated for 120 mins when diluted to 1, 0.1 and 0.01 mg/mL in HEPES, and incubated at 1 mg/mL for 120, 60, 30, 20 and 10 mins. Images were obtained for up to two weeks after staining. p-H₈TPPA-iPr₈ and p-TBr₃PPA-iPr₂ were only incubated for 120 mins at 1 mg/mL, including an incubation with dimethyl sulfoxide (DMSO). Negative controls were included by incubating ribs with the buffers alone. Following incubation with the respective dyes, slides were washed with their respective buffers (3×5 mins) and counter stained with DAPI, diluted in PBS, for 20 mins. After washing with PBS (3×5 mins), slides were mounted with 70% glycerol, diluted in PBS. Images were acquired on a Leica DM5500 epifluorescent microscope, using a 20× objective. The exposure times used to capture these images were kept the same throughout imaging. Images were processed using the FIJI software.

### DESCRIPTION OF THE EXAMPLES

For this study, as seen in FIG. 1, we have synthesized 6 fluorescent probes. Three of them incorporate phenylphosphonic acid as HAP binding unit with highly conjugated BODIPY, and porphyrin cores, namely((4-(5,5-difluoro-1,3,7,9-tetramethyl-5H-414,514-dipyrrolo[1,2-c:2′,1′-f][1,3,2]diazaborinin-10-yl)phenyl)phosphonic acid) (BODIPY-PPA), 5,10,15,20-tetrakis[p-phenylphosphonicacid] porphyrin (p-H₈TPPA) and 5,10,15,20-tetrakis[m-phenylphosphonic acid] porphyrin (m-H₈TPPA) (FIG. 1).

Furthermore, all fluorophores listed in Table 1 were synthetized via previously known state of the art from the corresponding brominated precursors. These methods include Ni and Pd catalyzed synthesis of arylphosphonic acids, and Suzuki Cross Coupling Methods (see Schütrumpf, A. et al. “Tetrahedral Tetraphosphonic Acids. New Building Blocks in Supramolecular Chemistry.” Crystal Growth & Design 2015, 15 (10), 4925-4931; Schütrumpf, A. et al. “Synthesis of Some Di- and Tetraphosphonic Acids by Suzuki Cross-Coupling.” Zeitschrift für anorganische and allgemeine Chemie 2018, 644 (19), 1134-1142).

p-H₈TPPA has four para positioned PPA units and BODIPY-PPA has one para positioned PPA promoting direct conjugation between the HAP and the fluorescent core. On the other hand, m-H₈TPPA has four meta positioned PPA units creating a more protective environment between the fluorescent porphyrin core and the HAP. We then explored their potential for binding hydroxyapatite. As a control group we used three different phosphonate diesters p-H₈TPPA-iPr₈, 5,10,15-tris[p-bromophenyl]-20-[p-phenyl-, bis(1-methylethyl) ester phosphonic acid] porphyrin (p-TBr3PPA-iPr₂) with bulkyisopropyl groups, and BODIPY-PPA-2Et₂ with two ethyl groups to block the metal-phosphonate interactions. To the best of our knowledge, all of the fluorescent probes except the p-H₈TPPA and its ester form are novel, and have not previously appeared in the literature, and this study reports the first synthesis of a phosphonic acid with a BODIPY fluorescent core. [10, 19] Previous synthesis of p-H₈TPPA relied on Ni catalyzed Arbuzov reaction. Therefore, p-H₈TPPA that is synthesized using this route usually had Ni in the imidazole ring the porphyrin core. In this study, we have used an alternative method using Pd catalyzed Arbuzov reaction to obtain metal free p-H₈TPPA. Likewise, we provide two alternative methods to synthesize p-H₈TPPA and m-H₈TPPA. BODIPY-PPA was synthesized using diethyl (4-formylphenyl)phosphonate and and 2,4-Dimethylpyrrole.

To further probe their HAP binding properties, we have used laterally sectioned mouse ribs as an example of HAP, which were incubated with p-H₈TPPA at concentrations ranging from 0.01 to 1.0 mg/mL in HEPES buffer for varying times at room temperature and imaged by fluorescence microscopy (for details see description of the figures), to assess HAP binding capability. Ribs incubated with 1 mg/mL meso-tetra(4-phosphorylphenyl)porphine showed a greater fluorescent intensity than both 0.1 mg/mL and 0.01 mg/mL (FIG. 2A-C, respectively), whilst still showing clear fluorescent signal at a concentration of 0.1 mg/mL. This indicates that the dye binds to HAP with a concentration dependence in the range tested.

When the mouse ribs were incubated with 1 mg/mL para-tetra(4-phosphorylphenyl)porphine in HEPES for differing times, 10 to 120 mins, there was a clear increase in fluorescence intensity with an increasing incubation time (FIG. 4A-E). Although there was an increase in fluorescence intensity, there was still a clear fluorescent signal when the ribs were incubated for 10 mins compared to the HEPES only negative control (FIG. 4E compared to FIG. 4F).

When ribs were incubated with p-H₈TPPA diluted to 1 mg/mL in different buffers, there was no clear difference between the fluorescence intensity of HEPES, PBS, TBS (FIG. 3A-C, respectively). However, when p-H₈TPPA was diluted in dH₂O (FIG. 3D), there was no fluorescent signal. Images were also obtained for the two weeks following the original staining.

There was a noticeable decrease in fluorescence intensity between ribs incubated with 1 mg/mL p-H₈TPPA in HEPES buffer and imaged on the day of staining compared with an image taken after 7 days later (FIG. 5A and FIG. 51, respectively). Mouse ribs stained with p-H₈TPPA diluted in PBS and TBS both showed fluorescent signal above background signal one week after staining (PBS, FIG. 5B and FIG. 5J; TBS, FIG. 5C and FIG. 5K, respectively). After two weeks, there was still a weak fluorescent signal observed for p-H₈TPPA diluted in PBS, but not for the other solvents. (FIG. 5N compared to FIGS. 5M, O and P). No signal was associated with incubating the ribs with the solvents as a negative control (FIG. 5Q-T).

Phosphonic acid moieties in m-H₈TPPA are more protected compared to the para positioned p-H₈TPPA. Mouse ribs were incubated with m-H₈TPPA at different concentrations, 1 to 0.01 mg/mL in HEPES buffer. Ribs incubated with each concentration of m-H₈TPPA showed little to no difference in the fluorescent intensity between 1, 0.1 and 0.01 mg/mL (FIG. 6A-C, respectively).

When the mouse ribs were incubated with 1 mg/mL m-H₈TPPA in HEPES for differing times, 10 to 120 mins, there no clear difference in fluorescence intensity with increasing incubation times (FIG. 7A-E, respectively). No fluorescent signal was observed when the ribs were incubated with HEPES alone (FIG. 7F).

Images were also obtained for the two weeks following the original staining. However, unlike the para isomer (FIG. 5), there was no noticeable decrease in fluorescence intensity between ribs incubated with 1 mg/mL m-H₈TPPA in HEPES buffer and imaged on the day of staining compared with images taken 4, 7 and 14 days later (FIG. 8A-D, respectively). This could be associated with the more protected HAP phosphonic acid interaction in the meta position limiting the interactions with the surrounding molecules solvents etc. Again, no signal was associated with incubating the ribs with HEPES only as a negative control (FIG. 8E).

The red filter (λ_{ex/em} 578/603 nm) has been used to visualize the fluorescent signal of the dye bound to HAP in this channel (FIGS. 9, 10 and 11). The dye shows a strong concentration dependency of staining, with a noticeable increase in fluorescence intensity when incubating with 1 mg/mL BODIPY-PPA compared to either 0.1 mg/mL or 0.01 mg/mL (FIG. 9A-C, respectively).

A similar decrease in fluorescence intensity is observed for decreasing incubation times from 120 to 10 mins (FIG. 10A-E, respectively). No fluorescent signal was observed under these conditions when the slices were incubated with HEPES alone (FIG. 10F).

There appeared to be only a very slight decrease in fluorescence intensity of dye up to two weeks following staining, with no signal was associated with incubating the ribs with HEPES only as a negative control (FIG. 11A-E, respectively).

As seen in FIG. 3, the fluorescence intensity is increased upon HAP binding of the fluorescent probe with p-H₈TPPA having four phosphonic acid groups bonded to the sp2 carbons of the fluorescent core.

Comparable experiments were conducted using the phosphonateesters BODIPY-PPA-2Et₂, p-TBr3PPA-iPr₂ (see FIG. 12), p-H₈TPPA-iPr₈ (see FIG. 13) as stains. As seen by the lack of HAP-associated staining, it seems evident that the interaction of phosphonates with HAP was reduced by the isopropylester groups on the phosphonates, preventing binding and interaction of the fluorescent porphyrin core with the HAP. On the other hand, the phosphonatediester of the BODIPY-PPA showed some interaction with the HAP, perhaps because phosphonate oxygens were more available to interact with the HAP (See the crystal structure FIG. 1d).

Mouse ribs were incubated for 120 mins with p-TBr3PPA-iPr₂ (5,10,15-tris[p-bromophenyl]-20-[p-phenyl-, bis(1-methylethyl) ester phosphonic acid] porphyrin) diluted to 1 mg/mL in different buffers. It was observed that the dye precipitated in HEPES, PBS, TBS and dH₂O (FIG. 12A-D) during the incubation period. This resulted in a fluorescent signal from the precipitated dye crystals, with no binding to HAP occurring. An additional incubation with the dye in an apolar solvent, DMSO, was attempted but this also resulted in the precipitation of the dye (FIG. 12E). Negative controls showed the fluorescent signal excited with λₑₓ 595 nm was due to dye precipitation and not the buffer itself (FIG. 12F-J).

Mouse ribs were incubated for 120 mins with p-H₈TPPA-iPr₈ phosphonate diester diluted to 1 mg/mL in different buffers. Similar to the tribromoporphine dye, it was observed that the dye precipitated in HEPES, PBS, TBS and dH₂O (FIG. 13A-D) during the incubation period. This resulted in a fluorescent signal from the precipitated dye crystals, with no binding to HAP occurring. An additional incubation with the dye in an apolar solvent, DMSO, was attempted but this also resulted in the precipitation of the dye (FIG. 13E). Negative controls showed the fluorescent signal excited with Aex 595 nm was due to dye precipitation and not the buffer itself (FIG. 13F-J).

As seen in FIG. 14, the absorbance peak (FIG. 14A) of p-H₈TTPA in PBS, TBS and HEPES with the addition of HAP corresponds with the absorbance of the dye in d-DMSO. There is a noticeable shift in the absorbance peak maximum when diluted in d H₂O, from 415 nm to 435 nm, which suggests a shift in the fluorescent properties of the dye due to protonation. This is also reflected in the maximal peak of the fluorescence intensity (FIG. 14 B), with a shift from λₑₘ 646-648 nm for PBS, TBS and HEPES to λₑₘ 675 nm when diluted in dH₂O. The fluorescence intensity of p-H₈TPPA is also substantially increased by 1.8-, 2.0-, 2.0-, and 1.6-fold upon HAP binding of the fluorescent probe p-H₈TPPA in PBS, TBS, HEPES and d H₂O, respectively (FIG. 14 B, solid vs. dashed lines). Previously reported bisphosphonate fluorophores had spa aliphatic carbons between the fluorescent core and HAP binding, therefore, such systems merely functioned as fluorescent labels with no change in emission due to HAP binding since they lacked synergistic interaction between the HAP and the fluorescent core. Phosphonic acids that have direct sp² bonds to the fluorescent core could extend the conjugation of the fluorescent core to the HAP and thereby could initiate changes in the ground and excited states resulting in quenching or enhancing the fluorescence emission. As seen in FIG. 14 (B), upon binding of HAP, p-H₈TPPA produces increased fluorescence supporting this hypothesis; p-H₈TPPA is thus the first example of a single sp² bonded phosphonic acid unit targeting HAP in the literature.

Comparable experiments with mouse rib sections were conducted using the isopropyldiester forms p-H₈TPPA-iPr₈ and m-H₈TPPA-OEt₈ as stains. We found a complete lack of HAP-associated staining with these compounds suggesting that the interaction of phosphonates with HAP was reduced by the isopropylester groups on the phosphonates, preventing binding and interaction of the fluorescent porphyrin core with the HAP. We also noted that the presence of hydrophobic isopropyl groups dramatically reduced their solubilities in the aqueous.

In addition to the application on bone sections, the phenylphosphonic acid/esterified porphyrins' propensity for cellular uptake was tested on proliferating human cells (FIG. 15). Aiming to use phenylphosphonic acid functionalized porphyrins in in vivo applications, the negative charge on the deprotonated phosphonate might be a handicap by limiting cell permeability. Optional demasking of phosphonate esters in metabolic surroundings might occur, recreating the metal binding PhPO₃²⁻. This possibility led us to examine p-H₈TPPA and p-H₈TPPA-iPr₈ suitability for in vivo cellular experiments by testing their permeability on proliferating human THP-1 monocytes.

We have treated THP-1 monocytes with the Phosphorylphenyl-modified porphyrins in loading buffer (10 mM HEPES, pH 7.35, 120 mM NaCl, 5.4 mM KCl, 5 mM glucose, 1.3 mM CaCl₂, 1 mM MgCl₂, 1 mM NaH₂PO₄, 0.3% bovine serum albumin) for 60 min. Excess fluorescence dye was removed by multiple washing steps before fluorescence emission scanning using 420 nm excitation (Tecan Infinite M200 reader; Tecan, Germany). (Haase et al. 2006) p-H₈TPPA was much more efficient in cell labeling compared to the isopropyl-modified molecule. We hypothesize that the phosphonates, being somewhat amphipathic, were able to penetrate the cell membrane, whereas the more nonpolar esters were not as efficient.

### CONCLUSION OF THE EXAMPLES

Herein, we report the synthesis of novel fluorescent probes namely BODIPY-PPA, p-H₈TPPA, m-H₈TPPA, BODIPY-PPA-2Et₂, p-TBr3PPA-iPr₂, p-H₈TPPA-iPr₈ in which the phosphonic acid metal binding unit(s) are exclusively bonded to sp2 carbon atoms of the fluorescent core. Sp2 bonding of the metal binding unit to the fluorescent core further extended the conjugation of the fluorescent core to the HAP leading significant increase in fluorescence upon HAP binding. We further used these fluorescent probes to target the HAP in mouse ribs and observed their interaction with human cells. The more protected metal binding nature of the m-H₈TPPA resulted in longer fluorescence period compared to the p-H₈TPPA. As a control, we have also synthesized phosphonatediesters of the synthesized fluorescent probes, which showed no HAP binding. p-H₈TPPA can travel through the cellular membrane, whereas the presence of bulky and hydrophobic isopropyl groups in p-H₈TPPA-iPr₈ hindered their passage through the cellular membrane. This study shows that compact fluorescent probes with phosphonic acid metal binding group(s) may be used to monitor microcalcifications, calcifications. In addition, their easy acceptance into the cellular matrix indicate that they could be used to target organelle specific calcifications such as mitochondrial calcifications. Reported low toxicity of arylphosphonic acid fluorophores and the new synthetic methods indicate that they can be used in targeting wide range of calcifications in vivo. We are currently developing our library to target organelle specific calcifications.

### REFERENCES

1. G. Yucesan et al./Coordination Chemistry Reviews 369 (2018) 105-122
2. Cancer Treatment Reviews 76 (2019) 57-67
3. X. Yu, J. R. Doroghazi, S. C. Janga, J. K. Zhang, B. Circello, B. M. Griffin, D. P. Labeda, W. W. Metcalf, Proc. Natl. Acad. Sci. U.S.A. 110 (2013) 20759-20764.
4. Huang, J., Su, Z. & Xu, Y. J MolEvol (2005) 61: 682. https://doi.org/10.1007/s00239-004-0349-4
5. Born et al., Science 358, 1336-1339 (2017)
6. ChemMedChem 2019, 14, 190-216
7. J. B. Rodriguez, B. N. Falcone, S. H. Szajnman, Expert Opin. Drug Discovery, 2016, 11, 307-320.
8. R. G. G. Russell, Bone 2011, 49, 2-19.
9. Mimicking cellular phospholipid bilayer packing creates predictable crystalline molecular metal-organophosphonate macrocycles and cages, AysunBulut, Maria Maares, KaanAtak, YunusZorlu, BünyeminÇoşut, Jon Zubieta, Jens Beckmann, Hajo Haase, Gündo\check{g}Yücesan, CrystEngComm, 2018, 20, 2152-2158
10. Alkali Phosphonate Metal Organic Frameworks, Maria Maares, M. MenafAyhan, Kai. B. Yu, A. OzgurYazaydin, KevserHarmandar, Hajo Haase, Jens Beckmann, YunusZorlu, and Gündo\check{g}Yücesan, Chem. Eur. J., 2019, 25, 11214-11217
11. R. Motekaitis, I. Murase, A. E. Martell, Inorg. Chem. 1976, 15, 2303.
12. T. K. Hurst, D. Wang, R. B. Thompson, C. A. Fierke, Biochim. Biophys. Acta Proteins Proteomics 2010, 1804, 393-403.
13. I. R. Salcedo, R. M. P. Colodrero, M. Bazaga-Garc_a, A. Vasileiou, M. Papadaki, P. Olivera-Pastor, A. Infantes-Molina, E. R. Losilla, G. Mezei, A. Cabeza, K. D. Demadis, CrystEngComm 2018, 20, 7648— 7658.
14. Angew. Chem. Int. Ed. 2014, 53, 10668-10672
15. Synthesis of Some Di- and Tetraphosphonic Acids by Suzuki Cross-Coupling, Alexandra Schütrumph, Andrew Duthie, Enno Lork, Gündo\check{g} Yücesan, Jens Beckmann, ZAAC, 2018, 644 (19), 1134-1142
16. Tetrahedral Tetraphosphonic Acids. New Building Blocks in Supramolecular Chemistry Alexandra Schütrumpf, Erdo\check{g}anKirpi, AysunBulut, Flavien L. Morel, Marco Ranocchiari, Enno Lork, YunusZorlu Simon Grabowsky, Gündo\check{g} Yücesan, and Jens Beckmann, Cryst. Growth & Design 2015, 15, 4925-4931
17. A novel fluorescein-bisphosphonate based diagnostic tool for the detection of hydroxyapatite in both cell and tissue models, (2018) 8:17360|DOI:10.1038/s41598-018-35454-9
18. doi:10.1038/bonekey.2014.24
19. www.pnas.org/cgi/doi/10.1073/pnas.1509310112

1. A fluorescent probe comprising
one or more metal binding functional group, preferably selected from the group comprising phosphonic acid group and arsonic acid group,
and a fluorescent core,
wherein the one or more functional group is covalently linked to a sp²-carbon atom of the fluorescent core.

2. The fluorescent probe according to claim 1, wherein the fluorescent core is an organic fluorescent compound/moiety.

3. The fluorescent probe according to claim 1, wherein the one or more metal binding functional group is a phosphonic acid group.

4. The fluorescent probe according to claim 1, wherein the probe comprises at least two, three, four, five, six, seven, eight, nine or ten or more metal binding functional groups.

5. The fluorescent probe according to claim 1, wherein the compound is selected from the group consisting of
((4-(5,5-difluoro-1,3,7,9-tetramethyl-5H-414,514-dipyrrolo[1,2-c:2′,1′-f][1,3,2]diazaborinin-10-yl)phenyl)phosphonic acid) (BODIPY-PPA),
5,10,15,20-tetrakis[p-phenylphosphonic acid] porphyrin (p-H₈TPPA), and
5,10,15,20-tetrakis[m-phenylphosphonic acid] porphyrin (m-H₈TPPA).

6. The fluorescent probe according to claim 1, wherein the probe can bind to calcium and/or a calcification.

7. The fluorescent probe according to claim 1, wherein binding of the probe to calcium, a calcification or HAP leads to an increase in fluorescence.

8. A contrast agent, comprising a fluorescent probe according to claim 1.

9. The fluorescent probe according to claim 1 for use in a method of detecting calcium in a bodily tissue.

10. The fluorescent probe for use according to claim 9, wherein the method is a diagnostic method.

11. The fluorescent probe for use according to claim 9, wherein the method is for detecting
bone growth and/or bone resorption,
a soft tissue calcification, and/or
for early diagnosis of breast cancer.

12. The fluorescent probe for use according to claim 9, wherein the method is for imaging tissue, lumens, or cells, the method comprising
contacting the tissue, lumen or cell with a fluorescent probe according to any one of claims 1-7,
irradiating the tissue, lumen, or cells at a wavelength absorbed by the compound;
and detecting a signal from the fluorescent probe, thereby imaging the tissue, lumen, or cells.

13. The fluorescent probe for use in a method according to claim 12, wherein the fluorescent probe is administered to a subject comprising the tissue, lumen, or cells.

14. Use of a fluorescent probe according to claim 1, for detecting calcium, a calcification and/or HAP.

15. The use of a fluorescent probe according to claim 14, wherein the calcium, calcification of HAP is deposited in a bodily tissue.

16. The fluorescent probe according to claim 2, wherein the organic fluorescent compound/moiety is a tetrapyrrole derivative.

17. The fluorescent probe according to claim 16, wherein the tetrapyrrole derivative is one or more of a porphyrin or phthalocyanine, an acridine, BODIPY, a cyanine or cyanine derivative, a carbazole, a coumarine or coumarine derivative, or a xanthene or xanthene derivative.

18. The fluorescent probe according to claim 17, wherein the xanthene or xanthene derivative is fluorescein or rhodamine.

19. The fluorescent probe according to claim 6, wherein the calcium and/or a calcification is hydroxyapatite (HAP).

20. The fluorescent probe for use according to claim 9, wherein the diagnostic method comprises
administering the fluorescent probe to a subject, or
exposing an isolated sample to the fluorescent probe in vitro.
